(19) United States
(12) Patent Application Publication
(10) Pub. No.: US 2020/0046696 A1
(43) Pub. Date: Feb. 13, 2020

(54) TREATMENT OF CANCER WITH SMG1-INHIBITORS

(71) Applicant: Celgene Corporation, Summit, NJ (US)

(72) Inventor: Ellen Filvaroff, San Francisco, CA (US)

(21) Appl. No.: 16/485,517

(22) PCT Filed: Feb. 13, 2018
(86) PCT No.: PCT/US18/17970

§ 371 Date: Aug. 13, 2019

Related U.S. Application Data
(60) Provisional application No. 62/458,837, filed on Feb. 14, 2017.

(51) Int. Cl. A61K 31/4985; G01N 33/574; A61P 35/00

(52) U.S. Cl. CPC A61K 31/4985; G01N 33/57496; G01N 2500/04; G01N 2800/52; A61P 35/00

(57) ABSTRACT

Provided herein are methods of treating NMD-dependent tumors by administering an SMG1-inhibitor to a patient in need of such treatment and combination therapies comprising the same.

This application claims the benefit of U.S. Provisional Application No. 62/458,837, filed Feb. 14, 2017, the entire contents of which are incorporated herein by reference.

### 1. FIELD

Provided herein are methods for treating or preventing an NMD-dependent tumor by administering an NMD-inhibitor or an SMG1-inhibitor, such as for example, Compound 1.

### 2. BACKGROUND

Kinases play a vital role in driving oncogenic pathways and have been the mainstay in the development of therapeutics across multiple cancers (Rask-Andersen, M., et al., Advances in kinase targeting: current clinical use and clinical trials. Trends Pharmacol Sci, 2014. 35(11): p. 604-20; Zhang, J., P. L. Yang, and N. S. Gray, Targeting cancer with small molecule kinase inhibitors. Nat Rev Cancer, 2009. 9(1): p. 28-39).

Nonsense-mediated mRNA decay (NMD) is a mammalian translation-coupled mechanism that selectively degrades mRNAs containing premature translation-termination (nonsense) codons. If mRNAs are not degraded, such mRNAs can result in truncated proteins with dominant-negative or deleterious gain-of-function activities. These truncated proteins can lead to various diseases and conditions. NMD has also been linked to pre-mRNA splicing.

Because deregulation of the cellular response to various types of stress can contribute to tumor growth and in certain cases contribute to resistance to chemotherapy, inhibiting the NMD pathway presents one option for cancer therapies. Accordingly there is a need in the art for inhibitors of NMD and SMG1 for treatments for a variety of diseases, including cancers. Provided herein are solutions to these problems and others in the art.

Citation or identification of any reference in Section 2 of this application is not to be construed as an admission that the reference is prior art to the present application.

### 3. SUMMARY

In a first aspect provided herein is a method for treating or preventing an NMD-dependent tumor, the method comprising administering an effective amount of an NMD-inhibitor to a patient having an NMD-dependent tumor.

In one embodiment, the NMD-dependent tumor is an SMG1-dependent tumor.

In one embodiment, the NMD-inhibitor is an SMG1-inhibitor.

In one embodiment, the SMG1-inhibitor is 1-ethyl-7-(2-methyl-6-(1H-1,2,4-triazol-3-yl)pyridin-3-yl)-3,4-dihydropyrazino[2,3-b]pyrazin-2(1H)-one, a tautomer, solvate, hydrate, co-crystal, clathrate, polymorph, or pharmaceutically acceptable salt thereof. In one embodiment, the SMG1-inhibitor is 1-ethyl-7-(2-methyl-6-(1H-1,2,4-triazol-3-yl)pyridin-3-yl)-3,4-dihydropyrazino[2,3-b]pyrazin-2(1H)-one. In one embodiment, the SMG1-inhibitor is a pharmaceutically acceptable salt of 1-ethyl-7-(2-methyl-6-(1H-1,2,4-triazol-3-yl)pyridin-3-yl)-3,4-dihydropyrazino[2,3-b]pyrazin-2(1H)-one. In one embodiment, the SMG1-inhibitor is a solvate of 1-ethyl-7-(2-methyl-6-(1H-1,2,4-triazol-3-yl)pyridin-3-yl)-3,4-dihydropyrazino[2,3-b]pyrazin-2(1H)-one. In one embodiment, the SMG1-inhibitor is a hydrate of 1-ethyl-7-(2-methyl-6-(1H-1,2,4-triazol-3-yl)pyridin-3-yl)-3,4-dihydropyrazino[2,3-b]pyrazin-2(1H)-one. In one embodiment, the SMG1-inhibitor is a polymorph (including polymorphic salts thereof) of 1-ethyl-7-(2-methyl-6-(1H-1,2,4-triazol-3-yl)pyridin-3-yl)-3,4-dihydropyrazino[2,3-b]pyrazin-2(1H)-one.

In one embodiment, the NMD-dependent tumor is characterized by modulation of activity or expression of one or more splicing factors. In a further embodiment, the modulation is inhibition. In another embodiment, the modulation is the result of a mutation.

In one embodiment, the NMD-dependent tumor is a breast tumor, ovarian tumor, papillary thyroid tumor, perivascular epithelioid cell tumor (PEComa), renal tumor, adenocystic carcinoma, chronic lymphocytic leukemia (CLL), myelodysplastic syndrome (MDS), skin melanoma, uveal melanoma, T-ALL, acute myelogenous leukemia (AML), chronic myelomonocytic leukemia (CMML), myelofibrosis, essential thrombocythemia, multiple myeloma, lung tumor, pancreatic tumor, prostate tumor, Wilms tumor, or glioblastoma. In one embodiment, the NMD-dependent tumor is prostate cancer, for example, castration-resistant prostate cancer. In another embodiment, the NMD-dependent tumor is multiple myeloma. In yet another embodiment, the NMD-dependent tumor is lung cancer, for example, non-small cell lung cancer. In one embodiment, the NMD-dependent tumor is myeloproliferative neoplasm (MPN).

In another aspect provided herein is a method for achieving one or more clinical endpoints associated with treating or preventing an NMD-dependent or SMG1-dependent tumor described herein.

In one embodiment, a patient described herein can show a positive tumor response, such as inhibition of tumor growth or a reduction in tumor size.

In one embodiment, a patient described herein can achieve a Response Evaluation Criteria in Solid Tumors (for example, RECIST 1.1) of complete response, partial response or stable disease after administration of an effective amount of Compound 1 (or a combination therapy described herein).

In one embodiment, a patient described herein can show increased survival without tumor progression.

In one embodiment, a patient described herein can show inhibition of disease progression, inhibition of tumor growth, reduction of primary tumor, relief of tumor-related symptoms, inhibition of tumor secreted factors (including tumor secreted hormones, such as those that contribute to carcinoid syndrome), delayed appearance of primary or secondary tumors, slowed development of primary or secondary tumors, decreased occurrence of primary or secondary tumors, slowed or decreased severity of secondary effects of disease, arrested tumor growth and regression of tumors, increased Time To Progression (TTP), increased Progression Free Survival (PFS), and/or increased Overall Survival (OS), among others.

In another embodiment, are methods for increasing the overall survival, objective response rate, time to progression, progression-free survival and/or time-to-treatment failure of a patient having an NMD-dependent or SMG1-dependent tumor described herein comprising administering an effective amount of an NMD-inhibitor or SMG1-inhibitor (e.g., Compound 1) as described herein.

In yet another aspect provided herein is a method for decreasing mortality of a patient having an NMD-dependent or SMG1-dependent tumor described herein comprising administering an effective amount of an NMD-inhibitor or SMG1-inhibitor (e.g., Compound 1) as described herein.

In another aspect provided herein is a method of testing for responsiveness to an NMD-inhibitor in a patient, the method comprising screening a biological sample obtained from the patient for the presence of an NMD-marker, wherein the presence of the NMD-marker indicates an increased likelihood that the patient will be responsive to treatment with the NMD-inhibitor.

In still another aspect provided herein is a method of testing for responsiveness to an SMG1-inhibitor for treating or preventing an SMG1-dependent tumor in a patient, the method comprising screening a biological sample obtained from the patient for the presence of an SMG1-marker, wherein the presence of the SMG1-marker indicates an increased likelihood that the patient will be responsive to treatment with the SMG1-inhibitor.

In yet another aspect provided herein is a method for testing for responsiveness to an SMG1-inhibitor administered to a patient for treatment or prevention of an NMD-dependent tumor or an SMG1-dependent tumor, the method comprising:
(a) measuring a level of expression or activity of an SMG1-marker in a biological sample obtained from the patient;
(b) administering a dosage amount of the SMG1-inhibitor to the patient;
(c) measuring a level of expression or activity of the SMG1-marker in a second biological sample obtained from the patient after the administration of the dosage amount; and
(d) comparing the levels of expression or activity of the SMG1-marker from the first and second biological samples; (e.g., to determine inhibition)
wherein a modulation of the level of expression or activity of the SMG1-marker indicates an increased likelihood that the patient will be responsive to treatment with the SMG1-inhibitor.

Further provided herein is a combination therapy for treating or preventing an NMD-dependent tumor, the combination therapy comprising an SMG1-inhibitor, and one or more of an immuno-oncology treatment, a proteasome inhibitor, a premature termination codon (PTC) read-through compound, an NMD-inhibitor, or a splice factor inhibitor.

In one embodiment, the SMG1-inhibitor is 1-ethyl-7-(2-methyl-6-(1H-1,2,4-triazol-3-yl)pyridin-3-yl)-3,4-dihydropyrazino[2,3-b]pyrazin-2(1H)-one or a tautomer or pharmaceutically acceptable salt thereof.

### 4. BRIEF DESCRIPTION OF THE DRAWINGS

FIGS. 1A, 1B, 1C and 1D illustrate dose-response growth curves for the isogenic DNA-PK knockout cell lines HCT116/HCT116 DNA-PK−/− and M059K/M059J DNA-PK−/− treated with Compounds 1 and 2 as described herein. Compound 1 is more potent than Compound 2 in DNA-PK−/− cell lines suggesting that Compound 1 inhibits more than just TORK and DNA-PK.

FIGS. 2A and 2B illustrate results from ActivX KiNativ analysis (% change in Mass Spectrometry (MS) signal) comparing different doses of Compound 1 and Compound 2 treatment in four different cell lines suggesting Compound 1 binds (as measured by % inhibition in FIGS. 2A and 2B) 3 kinases in the PIKK family, TORK, DNA-PK and SMG1. FIG. 2A shows binding results for 0.3 μM dose. FIG. 2B shows binding results for 1.0 μM dose.

FIGS. 3A and 3B illustrate that Compound 1 inhibits SMG1 while Compound 2 does not appear to. FIG. 3A shows Western blot analysis for markers of TORK inhibition (pS6 and p4EBP1) and SMG1 inhibition (pATM/ATR substrate band colocalizing with UPF1) in isogenic HCT-116 cells. Markers of TORK inhibition (pS6 and p4EBP1) are reduced by Compounds 1 and 2. One marker of SMG1 inhibition (pATM/ATR substrate) is reduced by Compound 1 but not by Compound 2. FIG. 3B shows qPCR analysis on HCT-116 parental cells treated with Compound 1 and Compound 2. qPCR to normalized control gene HPRT1 and relative to DMSO. *=NMD Transcript. Treatment with Compound 1 increases levels of NMD transcripts while Compound 2 does not.

FIGS. 4A, 4B, 4C and 4D illustrate that treatment of HCT-116 cells with SMG1 small interfering RNA (siRNA) reduces SMG1 expression and pUPF1, and increases levels of NMD transcripts. FIG. 4A shows an SMG1 western blot confirming siRNA knock-down of SMG1 in HCT-116 parental cells. FIG. 4B shows a Western blot analysis of UPF1 and phosphorylation of UPF1 in HCT-116 cells transfected with siSMG1. FIG. 4C shows qPCR analysis of NMD transcripts in siSMG1 transfected HCT-116 parental cells. FIG. 4D shows qPCR analysis of NMD transcripts in siSMG1 transfected HCT-116 mutant cells. qPCR to normalized control gene HPRT1 and relative to siGAPDH. *=NMD Transcript.

FIGS. 5A and 5B illustrate that Compound 1, but not Compound 2, upregulates mRNA (FIG. 5A) and protein (FIG. 5B) expression of p53 mutant in Calu6 cells, as measured by RNA level (by RT-PCR) or protein level (by western blot). pUPF1 was evaluated at the protein level by western blotting with an antibody to the pATM/ATR substrate.

FIGS. 6A, 6B and 6C illustrate that Compound 1 inhibits SMG1 in vivo. FIG. 6A shows a Western blot analysis on HCT-116 xenograft tumors treated with Vehicle or Compound 1 Compound 1 for DNA-PK and SMG1 inhibition. FIG. 6B shows quantitation of Western blots, averaging 4 tumors per condition. Error bars represent standard deviation (SD). FIG. 6C shows qPCR analysis on HCT-116 xenograft tumors treated with vehicle or Compound 1. qPCR relative to control gene HPRT1 and normalized to Vehicle. Bars represent average for 4 tumors and error bars represent SD. *=NMD Transcript.

### 5. DETAILED DESCRIPTION

### 5.1 Definitions

All patents, applications, published applications and other publications are incorporated by reference in their entirety. Unless defined otherwise, all technical and scientific terms used herein have the same meaning as commonly understood by those of ordinary skill in the art to which the invention belongs.

Any methods, devices and materials similar or equivalent to those described herein are used in the practice of this invention. The following definitions are provided to facilitate understanding of certain terms used frequently herein and are not meant to limit the scope of the present disclosure. In the event that there is a plurality of definitions for a term herein, those in this section prevail unless stated otherwise. Headings used herein are for organizational purposes only and in no way limit the embodiments described herein.

The term “Compound 1” refers to a compound having the structure:

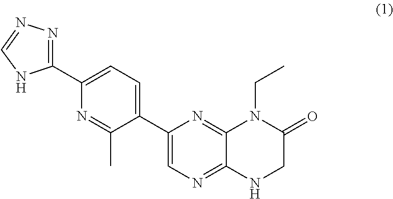 [SMILES: CCN1C(=O)CNc2ncc(-c3ccc(-c4nnc[nH]4)nc3C)nc21]

and having the alternative names of: 1-ethyl-7-(2-methyl-6-(1H-1,2,4-triazol-3-yl)pyridin-3-yl)-3,4-dihydropyrazino[2,3-b]pyrazin-2(1H)-one; 1-ethyl-7-(2-methyl-6-(4H-1,2,4-triazol-3-yl)pyridin-3-yl)-3,4-dihydropyrazino[2,3-b]pyrazin-2(1H)-one; and 1-ethyl-7-(2-methyl-6-(1H-1,2,4-triazol-5-yl)pyridin-3-yl)-3,4-dihydropyrazino[2,3-b]pyrazin-2(1H)-one, including pharmaceutically acceptable salts, clathrates, hydrates, solvates, stereoisomers, tautomers, metabolites, polymorphs, isotopologues and prodrugs thereof.

In one embodiment, Compound 1 refers to a compound having the structure

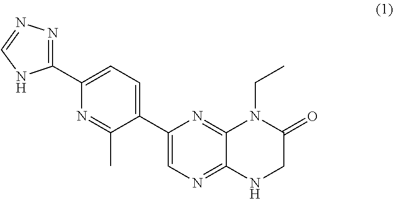 [SMILES: CCN1C(=O)CNc2ncc(-c3ccc(-c4nnc[nH]4)nc3C)nc21]

and having the alternative names of: 1-ethyl-7-(2-methyl-6-(1H-1,2,4-triazol-3-yl)pyridin-3-yl)-3,4-dihydropyrazino[2,3-b]pyrazin-2(1H)-one; 1-ethyl-7-(2-methyl-6-(4H-1,2,4-triazol-3-yl)pyridin-3-yl)-3,4-dihydropyrazino[2,3-b]pyrazin-2(1H)-one; and 1-ethyl-7-(2-methyl-6-(1H-1,2,4-triazol-5-yl)pyridin-3-yl)-3,4-dihydropyrazino[2,3-b]pyrazin-2(1H)-one, or a tautomer, solvate, hydrate, co-crystal, clathrate, polymorph, or pharmaceutically acceptable salt thereof.

In one embodiment, Compound 1 refers to a compound having the structure:

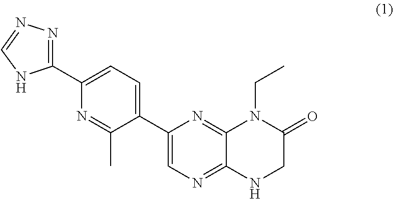 [SMILES: CCN1C(=O)CNc2ncc(-c3ccc(-c4nnc[nH]4)nc3C)nc21]

and having the alternative names of: 1-ethyl-7-(2-methyl-6-(1H-1,2,4-triazol-3-yl)pyridin-3-yl)-3,4-dihydropyrazino[2,3-b]pyrazin-2(1H)-one; 1-ethyl-7-(2-methyl-6-(4H-1,2,4-triazol-3-yl)pyridin-3-yl)-3,4-dihydropyrazino[2,3-b]pyrazin-2(1H)-one; and 1-ethyl-7-(2-methyl-6-(1H-1,2,4-triazol-5-yl)pyridin-3-yl)-3,4-dihydropyrazino[2,3-b]pyrazin-2(1H)-one, or a pharmaceutically acceptable salt or tautomer thereof.

Compound 1 can be prepared, for example, as described in U.S. Pat. No. 8,569,494, which is herein incorporated by reference in its entirety and for all purposes.

“Compound 2” refers to a compound having the structure:

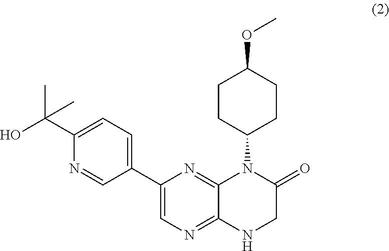 [SMILES: CO[C@H]1CC[C@H](N2C(=O)CNc3ncc(-c4ccc(C(C)(C)O)nc4)nc32)CC1]

and having the alternative names of: 7-(6-(2-hydroxypropan-2-yl)pyridin-3-yl)-1-((trans)-4-methoxycyclohexyl)-3,4-dihydropyrazino[2,3-b]pyrazin-2(1H)-one; 7-(6-(2-hydroxypropan-2-yl)pyridin-3-yl)-1-((1r,4r)-4-methoxycyclohexyl)-3,4-dihydropyrazino[2,3-b]pyrazin-2(1H)-one; and 7-(6-(2-hydroxypropan-2-yl)pyridin-3-yl)-1-((1R*,4R*)-4-methoxycyclohexyl)-3,4-dihydropyrazino[2,3-b]pyrazin-2(1H)-one, including pharmaceutically acceptable salts, clathrates, hydrates, solvates, stereoisomers, tautomers, metabolites, polymorphs, isotopologues and prodrugs thereof.

As used herein, the term “pharmaceutically acceptable salt(s)” refers to a salt prepared from a pharmaceutically acceptable non-toxic acid or base including an inorganic acid and base and an organic acid and base. Suitable pharmaceutically acceptable base addition salts of the SMG1-inhibitors described herein (e.g., Compound 1) include, but are not limited to metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine) and procaine. Suitable non-toxic acids comprise, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids comprise hydrochloric, hydrobromic, phosphoric, sulfuric, and methanesulfonic acids. Examples of specific salts thus include hydrochloride and mesylate salts. Others are well-known in the art, see for example, Remington's Pharmaceutical Sciences, 18^{th} eds., Mack Publishing, Easton Pa. (1990) or Remington: The Science and Practice of Pharmacy, 19^{th} eds., Mack Publishing, Easton Pa. (1995).

As used herein and unless otherwise indicated, the term “clathrate” means an NMD-inhibitor or an SMG1-inhibitor (e.g., Compound 1), or a salt thereof, in the form of a crystal lattice that contains spaces (e.g., channels) that have a guest molecule (e.g., a solvent or water) trapped within or a crystal lattice wherein an NMD-inhibitor is a guest molecule.

As used herein and unless otherwise indicated, the term “solvate” means an NMD-inhibitor or an SMG1-inhibitor (e.g., Compound 1), or a salt thereof, that further comprises a stoichiometric or non-stoichiometric amount of a solvent bound by non-covalent intermolecular forces. In one embodiment, the solvate is a hydrate.

As used herein and unless otherwise indicated, the term “hydrate” means an NMD-inhibitor or an SMG1-inhibitor (e.g., Compound 1), or a salt thereof, that further comprises a stoichiometric or non-stoichiometric amount of water bound by non-covalent intermolecular forces.

As used herein and unless otherwise indicated, the term “prodrug” means an NMD-inhibitor or an SMG1-inhibitor (e.g., Compound 1) derivative that can hydrolyze, oxidize, or otherwise react under biological conditions (in vitro or in vivo) to provide an active compound, particularly an NMD-inhibitor or SMG1-inhibitor as described herein. Examples of prodrugs include, but are not limited to, derivatives and metabolites of an NMD-inhibitor that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. In certain embodiments, prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger's Medicinal Chemistry and Drug Discovery 6^{th} ed. (Donald J. Abraham ed., 2001, Wiley) and Design and Application of Prodrugs (H. Bundgaard ed., 1985, Harwood Academic Publishers Gmfh).

As used herein and unless otherwise indicated, the term “stereoisomer” or “stereomerically pure” means one stereoisomer of an NMD-inhibitor or an SMG1-inhibitor (e.g., Compound 1) that is substantially free of other stereoisomers of that compound. For example, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The NMD-inhibitors described herein useful in the invention can have chiral centers and can occur as racemates, individual enantiomers or diastereomers, and mixtures thereof. All such isomeric forms are comprised within the embodiments disclosed herein, including mixtures thereof. The use of stereomerically pure forms of such NMD-inhibitors, as well as the use of mixtures of those forms is encompassed by the embodiments disclosed herein. For example, mixtures comprising equal or unequal amounts of the enantiomers of a particular NMD-inhibitor may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, N Y, 1962); and Wilen, S. H., Tables of Resolving Agents and Optical Resolutions p. 268 (E. L. Eliel, Ed., Univ. of Notre Dame Press, Notre Dame, Ind., 1972).

It should also be noted the NMD-inhibitors can comprise E and Z isomers, or a mixture thereof, and cis and trans isomers or a mixture thereof. In certain embodiments, the NMD-inhibitors are isolated as either the cis or trans isomer. In other embodiments, the NMD-inhibitors are a mixture of the cis and trans isomers.

“Tautomers” refers to isomeric forms of a compound that are in equilibrium with each other. The concentrations of the isomeric forms will depend on the environment the Compound 1 is found in and may be different depending upon, for example, whether the Compound 1 is a solid or is in an organic or aqueous solution. For example, in aqueous solution, pyrazoles may exhibit the following isomeric forms, which are referred to as tautomers of each other:

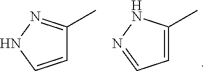

As readily understood by one skilled in the art, a wide variety of functional groups and other structures may exhibit tautomerism and all tautomers of the NMD-inhibitors or the SMG1-inhibitors (e.g., Compound 1) described herein are within the scope of the present invention.

It should also be noted NMD-inhibitors and SMG1-inhibitors described herein (e.g., Compound 1) can contain unnatural proportions of atomic isotopes at one or more of the atoms. For example, the compounds may be radiolabeled with radioactive isotopes, such as for example tritium (³H), iodine-125 (¹²⁵I) sulfur-35 (³⁵S), or carbon-14 (¹⁴C), or may be isotopically enriched, such as with deuterium (²H), carbon-13 (¹³C), or nitrogen-15 (¹⁵N). As used herein, an “isotopologue” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically enriched compounds are useful as therapeutic agents, e.g., cancer and inflammation therapeutic agents, research reagents, e.g., binding assay reagents, and diagnostic agents, e.g., in vivo imaging agents. All isotopic variations of the NMD-inhibitors or the SMG1-inhibitors (e.g., Compound 1) as described herein, whether radioactive or not, are intended to be encompassed within the scope of the embodiments provided herein. In some embodiments, there are provided isotopologues of the NMD-inhibitors or the SMG1-inhibitors (e.g., Compound 1), for example, the isotopologues are deuterium, carbon-13, or nitrogen-15 enriched NMD-inhibitors.

“Treating” as used herein, means alleviation, in whole or in part, of an NMD-dependent cancer, or a symptom thereof, or slowing, or halting of further progression or worsening of an NMD-dependent cancer.

“Responsiveness” or “responsive” when used in reference to a treatment refers to the degree of effectiveness of the treatment in lessening or decreasing the symptoms of a disease, disorder, or condition being treated. For example, the term “increased responsiveness” when used in reference to a treatment of a patient refers to an increase in the effectiveness in lessening or decreasing the symptoms of the disease when measured using any methods known in the art.

“Preventing” as used herein, means the prevention of the onset, recurrence or spread, in whole or in part, of an NMD-dependent cancer or an SMG1-dependent cancer, or a symptom thereof.

The term “effective amount” in connection with an NMD-inhibitor or an SMG1-inhibitor (e.g., Compound 1) described herein means an amount capable of alleviating, in whole or in part, symptoms associated with an NMD-dependent cancer or an SMG1-dependent cancer, or slowing or halting further progression or worsening of those symptoms. The effective amount of the NMD-inhibitor or SMG1-inhibitor, for example in a pharmaceutical composition, may be at a level that will exercise the desired effect; for example, about 0.005 mg/kg of a subject's body weight to about 100 mg/kg of a patient's body weight in unit dosage for both oral and parenteral administration. As will be apparent to those skilled in the art, it is to be expected that the effective amount of an NMD-inhibitor or SMG1-inhibitor disclosed herein may vary depending on the severity of the indication being treated.

The terms “patient” and “subject” as used herein include an animal, including, but not limited to, an animal such as a cow, monkey, horse, sheep, pig, chicken, turkey, quail, cat, dog, mouse, rat, rabbit or guinea pig, in one embodiment a mammal, in another embodiment a human. In one embodiment, a “patient” or “subject” is a human having an NMD-dependent cancer or an SMG1-dependent cancer described herein.

As used herein, and unless otherwise specified, the terms “cancer” and “tumor” are used interchangeably and refer to or describe the physiological condition in mammals that is typically characterized by unregulated cell growth. Examples of cancer comprise solid tumors and hematological cancers. In some embodiments, the cancer is a primary cancer, in others, the cancer is metastasized. A “NMD-dependent tumor” refers to a tumor characterized or caused by aberrant activity or expression of an NMD component or NMD pathway.

As used herein “solid tumors” includes, but is not limited to, bladder cancer (including, but not limited to, superficial bladder cancer), breast cancer (including, but not limited to, luminal B type, ER+, PR+ and Her2+ breast cancer), central nervous system cancer (including, but not limited to, glioblastoma multiforme (GBM), glioma, medulloblastoma, and astrocytoma), colorectal cancer, gastrointestinal cancer (including, but not limited to, stomach cancer, oesophagus cancer, and rectum cancer), endocrine cancer (including, but not limited to, thyroid cancer, and adrenal gland cancer), eye cancer (including, but not limited to, retinoblastoma), female genitourinary cancer (including, but not limited to, cancer of the placenta, uterus, vulva, ovary, cervix), head and neck cancer (including, but not limited to, cancer of the pharynx, oesophagus, and tongue), liver cancer, lung cancer (including, but not limited to, non-small cell lung cancer (NSCLC), small cell lung cancer (SCLC), mucoepidermoid, bronchogenic, squamous cell carcinoma (SQCC), and analplastic/NSCLC), skin cancer (including, but not limited to, melanoma, and SQCC), soft tissue cancer (including but not limited to, sarcoma, Ewing's sarcoma, and rhabdomyosarcoma), bone cancer (including, but not limited to, sarcoma, Ewing's sarcoma, and osteosarcoma), squamous cell cancer (including, but not limited to, lung, esophageal, cervical, and head and neck cancer), pancreas cancer, kidney cancer (including, but not limited to, renal Wilm's tumor and renal cell carcinoma), and prostate cancer. In one embodiment, the solid tumor is not triple negative breast cancer (TNBC). In some embodiments, the solid tumor is breast cancer, colon cancer, lung cancer or bladder cancer. In one such embodiment, the solid tumor is superficial bladder cancer. In another, the solid tumor is lung squamous cell carcinoma. In yet another embodiment, the solid tumor is luminal B type breast cancer.

As used herein “hematological cancer” includes, but is not limited to, leukemia (including, but not limited to, acute lymphocytic leukemia (ALL), chronic myeloid leukemia (CIVIL), acute T-cell leukemia, B cell precursor leukemia, acute promyelocytic leukemia (APML), plasma cell leukemia, myelomonoblastic/T-ALL, B myelomonocytic leukemia, erythroleukemia, and acute myeloid leukemia (AML)), lymphoma (including but not limited to Hodgkin's lymphoma, non-Hodgkin's lymphoma (NHL), Burkitt's lymphoma (BL), B cell lymphoma, lymphoblastic lymphoma, follicular lymphoma (FL), diffuse large B-cell lymphoma (DLBCL), large cell immunoblastic lymphoma), and multiple myeloma.

In the context of a cancer, inhibition may be assessed by inhibition of disease progression, inhibition of tumor growth, reduction of primary tumor, relief of tumor-related symptoms, inhibition of tumor secreted factors (including tumor secreted hormones, such as those that contribute to carcinoid syndrome), delayed appearance of primary or secondary tumors, slowed development of primary or secondary tumors, decreased occurrence of primary or secondary tumors, slowed or decreased severity of secondary effects of disease, arrested tumor growth and regression of tumors, increased Time To Progression (TTP), increased Progression Free Survival (PFS), increased Overall Survival (OS), among others. OS as used herein means the time from treatment onset until death from any cause. TTP as used herein means the time from treatment onset until tumor progression; TTP does not comprise deaths. As used herein, PFS means the time from treatment onset until tumor progression or death. In one embodiment, PFS rates will be computed using the Kaplan-Meier estimates. In the extreme, complete inhibition, is referred to herein as prevention or chemoprevention. In this context, the term “prevention” comprises either preventing the onset of clinically evident cancer altogether or preventing the onset of a preclinically evident stage of a cancer. Also intended to be encompassed by this definition is the prevention of transformation into malignant cells or to arrest or reverse the progression of premalignant cells to malignant cells. This comprises prophylactic treatment of those at risk of developing a cancer.

The terms “inhibition”, “inhibit”, “inhibiting” refer to a reduction in the activity, binding, or expression of a polynucleotide or a polypeptide or reduction or amelioration of a disease, disorder, or condition or a symptom thereof. Inhibiting as used here can include partially or totally blocking stimulation, decreasing, preventing, or delaying activation or binding, or inactivating, desensitizing, or down-regulating polynucleotide, protein or enzyme expression, activity or binding.

The term “administering” refers to the act of delivering a compound (e.g., an NMD- or SMG1-inhibitor) described herein into a subject by such routes as oral, mucosal, topical, suppository, intravenous, parenteral, intraperitoneal, intramuscular, intralesional, intrathecal, intranasal or subcutaneous administration. Parenteral administration comprises intravenous, intramuscular, intra-arteriole, intradermal, subcutaneous, intraperitoneal, intraventricular, and intracranial administration. Administration generally occurs after the onset of the disease, disorder, or condition, or its symptoms but, in certain instances, can occur before the onset of the disease, disorder, or condition, or its symptoms (e.g., administration for patients prone to such a disease, disorder, or condition). The term includes administering a cancer therapy as described herein.

The term “coadministration” refers to administration of two or more agents (e.g., an NMD- or SMG1-inhibitor described herein and one or more combination active agents as described herein). The timing of coadministration depends in part of the combination and compositions administered and can comprise administration at the same time, just prior to, or just after the administration of one or more additional therapies, for example cancer therapies such as chemotherapy, hormonal therapy, radiotherapy, or immunotherapy. NMD- and SMG1-inhibitors of the invention can be administered alone or can be coadministered to the patient. In some embodiments, coadministration is meant to include simultaneous or sequential administration of the Compound 1 individually or in combination (more than one compound or agent). Thus, the preparations can also be combined, when desired, with other active substances (e.g., to reduce metabolic degradation). The NMD- and SMG1-inhibitors described herein is used in combination with one another, with other combination active agents either known to be useful in treating a disease associated with cells expressing a particular kinase or as otherwise described herein, or with adjunctive agents that cannot be effective alone, but can contribute to the efficacy of the active agent.

“NMD-dependent cancer” and “NMD-dependent tumor” are used interchangeably herein and refer to tumors in which signaling polynucleotide(s), polypeptide(s), or other pathway(s) associated with the nonsense-mediated mRNA decay (NMD) pathway is/are dysregulated. Such cancers include, but are not limited to, solid tumors (such as gastric cancer, breast cancer, endometrial cancer, uterine cancer, colorectal cancer, synovial sarcoma, pancreatic cancer, melanoma, lobular carcinoma, prostate cancer, triple negative breast cancer (TNBC), non-small cell lung cancer (NSCLC), squamous cell lung carcinoma, lung adenocarcinoma, hepatocellular cancer (HCC), ovarian cancer, adenoid carcinoma, adrenocortical carcinoma, bladder/urothelial carcinoma, glioblastoma multiforme (GBM), cervical cancer, head and neck squamous cell carcinoma (HNSCC), kidney cancer, and thyroid cancer) and hematologic malignancies (such as acute myelogenous leukemia (AML), acute lymphoblastic leukemia (ALL), multiple myeloma (MM), chronic lymphocytic leukemia (CLL), chronic myelogenous leukemia (CIVIL)), as well as cancer stem cells in many tumors types, particularly those described herein. In one embodiment, the NMD-dependent tumor is prostate cancer, for example, castration-resistant prostate cancer. In another embodiment, the NMD-dependent tumor is multiple myeloma. In yet another embodiment, the NMD-dependent tumor is lung cancer, for example, non-small cell lung cancer.

“SMG1-dependent tumor” as used herein refers to tumors and cancers characterized by modulated expression or activity of SMG1, including variants, isoforms, and species homologs of human SMG1 (e.g., mouse). Such cancers include, but are not limited to, solid tumors (such as gastric cancer, breast cancer, endometrial cancer, uterine cancer, colorectal cancer, synovial sarcoma, pancreatic cancer, melanoma, lobular carcinoma, prostate cancer, triple negative breast cancer (TNBC), non-small cell lung cancer (NSCLC), squamous cell lung carcinoma, lung adenocarcinoma, hepatocellular cancer (HCC), ovarian cancer, adenoid carcinoma, adrenocortical carcinoma, bladder/urothelial carcinoma, glioblastoma multiforme (GBM), cervical cancer, head and neck squamous cell carcinoma (HNSCC), kidney cancer, and thyroid cancer) and hematologic malignancies (such as acute myelogenous leukemia (AML), acute lymphoblastic leukemia (ALL), multiple myeloma (MM), chronic lymphocytic leukemia (CLL), chronic myelogenous leukemia (CIVIL)), as well as cancer stem cells in many tumors types, particularly those described herein. In one embodiment, the SMG1-dependent tumor is prostate cancer, for example, castration-resistant prostate cancer. In another embodiment, the SMG1-dependent tumor is multiple myeloma. In yet another embodiment, the SMG1-dependent tumor is lung cancer, for example, non-small cell lung cancer.

A “NMD component” refers to a polynucleotide or polypeptide associated with the NMD pathway. Exemplary NMD components include those described throughout the present disclosure. In one embodiment, an NMD component is SMG1.

A “NMD-inhibitor” refers to agents which specifically and selectively inhibit one or more NMD components. NMD-inhibitors can include small molecule compounds (e.g., molecules less than about 1 kDa). In certain embodiments, NMD-inhibitors are compounds other than polynucleotides or polypeptides. In certain embodiments, an NMD-inhibitor is a compound described herein (e.g., an SMG1-inhibitor or Compound 1).

A “SMG1-inhibitor” refers to a moiety (e.g., compound, nucleic acid, polypeptide, antibody) that decreases, inhibits, blocks, abrogates or interferes with the activity or binding of serine/threonine-protein kinase SMG1 (e.g., nonsense mediated mRNA decay associated phosphatidylinositol 3-kinase-related kinase; SMG1; NCBI GI: 62243658) including variants, isoforms, and species homologs of human SMG1 (e.g., mouse). In certain embodiments, an SMG1-inhibitor comprises Compound 1.

As used herein, the term “immuno-oncology treatment” refers to molecules that totally or partially reduce, inhibit, interfere with or modulate one or more checkpoint proteins. Without being limited by a particular theory, checkpoint proteins appear to regulate T-cell activation or function. Numerous checkpoint proteins are known, such as CTLA-4 and its ligands CD80 and CD86; and PD-1 with its ligands PD-L1 and PD-L2 (Pardoll, Nature Reviews Cancer, 2012, 12, 252-264). These proteins appear responsible for co-stimulatory or inhibitory interactions of T-cell responses. Immune checkpoint proteins appear to regulate and maintain self-tolerance and the duration and amplitude of physiological immune responses. Immuno-oncology treatment includes antibodies and other molecules that are derived from antibodies, or other compounds that inhibit checkpoint proteins as provided above.

A “proteasome inhibitor” refers to a moiety (e.g., compound, nucleic acid, polypeptide, or antibody) that decreases, inhibits, blocks, abrogates or interferes with the activity or expression of one or more proteasomes, such as for example, 26S proteasome or the 20S proteasome. Non-limiting exemplary proteasome inhibitors comprise bortezomib, carfilzomib, Oprozomib, Ixazomib, Delanzomib, and Marizomib.

A “premature termination codon (PTC) read-through compound” refers to a moiety (e.g., compound, nucleic acid, polypeptide, or antibody) that suppresses nonsense premature termination codons (TGA, TAG, and TAA). In one embodiment, the PTC read-through compound is aminoglycoside antibiotic or analogue thereof. In one embodiment, the PTC read-through compound is G418.

A “splice factor inhibitor” refers to a moiety (e.g., compound, nucleic acid, polypeptide, or antibody) that decreases, inhibits, blocks, abrogates or interferes with the activity or expression of proteins associated with pre-mRNA splicing. In one embodiment, a splice factor inhibitor can inhibit a spliceosome. In one embodiment, a splice factor inhibitor can inhibit Srsf1.

A “Splicing factor” refers to proteins or protein complexes that function in splicing. Splicing factors comprise those that are required for constitutive splicing, and splicing of specific messages or groups of messages. As used herein the term also comprises SR proteins, which are known to be associated with constitutive pre-mRNA splicing.

An “NMD-marker” as used herein comprises mutations, copy number variations (CNV's, gains or losses), fusions, decreased/increased expression or mislocalization of miRNA, mRNA or protein, or changes in phosphorylation or activity of NMD-dependent genes or regulators. In one embodiment, an NMD-marker is an SMG1-marker.

An “SMG1-marker” as used herein comprises mutations, copy number variations (CNV's, gains or losses), fusions, decreased/increased expression or mislocalization of miRNA, mRNA or protein, or changes in phosphorylation or activity of SMG1 genes or regulators (such as, for example, 1110004F10Rik, 1110008L16Rik, 1700012D14Rik, 1700037H04Rik, 1810029B16Rik, 2310036022Rik, 2310037I24Rik, 2410004B18Rik, 2900064A13Rik, 4632411B12Rik, 5033414D02Rik, 9030025P20Rik, Aadac, AI314976, AI506816, Abhd12, Acin1, Acot8, Acox1, Actn4, Actr1a, Adss, Aes, Aga, Ahcyl1, Ak2, Akap8, Alg1, Alkbh1, Alkbh3, Alkbh6, Alpk1, Ankfy1, Ankmy1, Ap1b1, Ap2m1, Apobec1, Apoe, Aprt, Arfrp1, Arih2, Arpc1b, Arhgap11b, Ascc2, Atf4, Atf7, Atg2a, Atg3, Atp5j2, Atp6v0d1, Atp6v0e, Atp6v1c1, Atp6v1e1, Aup1, Azin1, BC031181, Bap1, Bcas3, Brd2, Brf2, Bod11, Brp441, Btbd1, Bub3, C2cd3, C6orf141, C6orf48, Ca5b, Cacnb1, Cald1, Camta2, Caprin1, Capns1, Cars2, Cbs, Ccdc111Ccdc130, Ccdc21, Ccnt2, Ccnb1ip1, Cdc37l1, Cdk2ap2, Cdkn2aip, Cfi, Cenpa, Cep250, Cept1, Cfb, Cf12, Chkb, Cinp, Clcn4-2, Clcn6, Clcn7, Cldn15, Clk1, Clock, Clptm1, Clta, Cops3, Cops5, Cops7b, Cox7c, Cpsf7, Creg1, Crlf2, Csnk1e, Csnk1g2, Ctage5, Ctla2a, Ctsa, Ctsd, Cxcl16, D10Jhu81e, D4Wsu53e, D6Wsu116e, Dazap1, Dci, Dclk2, Dclre1c, Ddit3, Ddx39, Ddx5, Dhrs3, Dhx15, Dis3, Dnajc25, Dnajc5, Dnajc7, Dnm2, Dpp7, Dpp8, Dscr3, Dtnbp1, Dusp11, Dusp12, Edc4, Edf1, Eef1a1, Eef1b2, Eef2, Eif3k, Eif4a2, Eif4g2, Ell2, Eno1, Epn1, Eprs, Eps1511, Erap2, Ergic1, Eri3, Erp44, Ewsr1, Exosc5, Fam13b, Fam105b, Fam125a, Fam149b, Fam45a, Fbxo18, Fis1, Flot1, Flot2, Foxk2, Fsd11, Fus, Fyb, Gadd45b, Gdf15, Gm15427, Gm3258, Gnl2, Gns, Golga4, Got1, Gp49a, Gpi1, Gramd1a, Grem1, Grn, Gsta3, Gtf2a2, H13, H19, H2-M3, H2-T22, H2afy, Hdac10, Hdgf, Haaol, Hexa, Hira, Hnrnpa1, Hnrnpc, Hnrnpd, Hnrnph1, Hnrnph3, Hnrnpk, Hnrnp1, Hnrp11, Hpca11, Hpn, Hps5, Hras1s, Hsf1, Hspa9, Hyou1, Idua, Ifi16, Ifi203, Ifrd1, Il4ra, Il24, Ilk, Ilkap, Irak1, Irf3, Itfg3, Itsn1, Jak1, Jmjd6, Kars, Khdrbs1, Krcc1, Lamp2, Lars, Lass2, Lats2, Ldlr, Lipg, Lgals1, Loh12cr1, Lpcat, Lsm6, Luc71, Ly6a, Map2k2, Mapkapk3, Matr3, Max, Mbd2, Mbnl1, Mbtps1, Mcm7, Mdm2, Mecr, Mettl21d, Mff, Mgea5, Mina, Mkln1, Mocs, Mocs1, Morf411, Mpdu1, Mpv1712, Mrpl13, Mrps10, Mrps18b, Mta1, Mtpap, Mtx1, Myolf, Naga, Napa, Napb, Nbpf14, Nbr1, Ncaph2, Ndufa7, Ndufa11, Ndufv3, Ndufb5, Ndufs6, Necap2, Nelf, Neu1, Ngf, Nisch, Nkiras1, Nktr, Nom1, Nosip, Nr1h3, Nrbp1, Nt5c2, Nt5c31, Nub1, Nubp2, Nudt9, Nup35, Nxt2, Ociad1, Ogfr, Pabpc4, Pam, Pan3, Papss2, Parp10, Parp6, Pcbp2, Pcmt1, Pcnx12, Pcsk7, Pctk3, Pcyt2, Pddc1, Pecam1, Peci, Pfdn5, Pfldb2, Phc3, Phf1, Phkg2, Pik3ap1, Pilrb1, Pion, Pkm2, Plekha1, Plxnb2, Pnpla7, Ppid, Ppie, Ppp1ca, Ppp1r15a, Ppp4r1, Preb, Prpf38b, Prpf40a, Prpf6, Prr3, Psma3, Psmg2, Ptbp1, Ptpb2, Ptges2, Pttg1ip, R3hdm1, Rab11a, Rab4a, Rabggtb, Rad52, Rassf1, Rbm25, Rbm3, Rbm6, Rbm7, Rce1, Rft1, Rfxank, Ripk1, Rnaseh2a, Rnaset2a, Rnd1, Rnf13, Rnf114, Rnf130, Rnf149, Rnf181, Rnps1, Rpl10a, Rpl12, Rpl13a, Rp11-706o15.1, Rpl17, Rpl3, Rpl37, Rps10, Rps12, Rps19, Rps19bp1, Rps3, Rps6, Rps6kc1, Rps9, Rpsa, Rufy2, Safb2, Sat1, Sdhaf2, Sec11c, Sec61a2, Secisbp2, Senp5, Setd3, Setd4, Setd5, Sidt1, Sf1, Sf3b1, Sf3b3, Srsf11, Srsf2, Srsf3, Srsf4, Srsf5, Srsf6, Srsf8, Srsf9, Sgms1, Sh2b3, Shmt2, Slc15a4, Slc25a12, Slc26a6, Slc40a1, Slc4a2, Slc7a6os, Slc9a8, Smcr71, Smek1, Smg5, Smndc1, Smox, Snrpa1, Snx4, Son, Spns1, Srsf2, Srrm1, Srrt, Stard3, Stom12, Stradb, Stx1a, Supv311, Surf1, Surf2, Tank, Tatdn1, Tbrg4, Tcirg1, Tecr, Tfip11, Thoc3, Thyn1, Tk2, Tm9sf4, Tmbim4, Tmbim6, Tmem11, Tmem149, Tmem156, Tmem183a, Tmem208, Tmem214, Tmem260, Tmem47, Tmem57, Tmem8, Tmem87a, Toe1, Tom112, Tomm34, Top3b, Tpcn1, Tpi1, Tpm1, Tpp2, Tpra1, Tprg1, Tpst2, Tra2a, Tra2b, Trmt1, Trmt6, Trpc1, Trub2, Tsc22d3, Tsg101, Tsku, Ttc33, Ttc39c, Tufm, Tug1, Txnrd1, Tyw5, U2af1, U2af2, Uap111, Ubap2, Ube2d3, Ube2k, Ube2n, Ub15, Ufc1, Ufsp2, Uggt1, phospho-Upf1, Upf2, Use1, Usp7, Vti1b, Vkorc111, Vps11, Vps29, Vps41, Wdfy2, Wdr451, Wdr82, Wdyhv1, Whamm, Xdh, Zc3h7a, Zcchc6, Zcrb1, Zdhhc16, Zdhhc3, Zfand2a, Zfand5, Zfp326, Zfp384, Zfp385a, Zfp523, Zfp553, Zfp655, Zfyve19, Zmat5, Znhit1, Zrsr2, or Zyx, or any combination thereof, which have been found to be associated herein with increased NMD activity and/or response to inhibition of SMG1.

“Modulation” refers to changing or varying a property or amount. For example, modulation refers changes by increasing or decreasing a property, such as the activity of a target molecule. Modulation can also refer to changes by increasing or decreasing a property, such as the expression or amount of a target molecule.

A “splicing regulator” “refers to proteins or protein complexes that function to control and regulate splicing (alternative splicing). Splicing regulators comprise those that are required for regulated splicing of specific messages or groups of messages.

Nonsense mediated RNA decay (NMD) is a surveillance system that reduces and/or eliminates mRNAs with premature translation-termination codons. It also can regulate levels of other RNAs. SMG1, a phosphoinositide 3-kinase related protein kinase (PIKK), appears to be required for NMD in vivo. Compounds described herein including, for example, Compound 1, inhibit SMG1. In other embodiments, compounds described herein including, for example, Compound 1 can inhibit also inhibit phosphatidylinositol 3-kinases (PIKKs) such as TORK and DNAPK.

### 5.2 Methods of Use

Provided herein are methods for treating or preventing an NMD-dependent tumor by administering an effective amount of an NMD-inhibitor described herein to a patient having an NMD-dependent tumor. In one aspect, the methods comprise treating an NMD-dependent tumor by administering an effective amount of an NMD-inhibitor described herein to a patient having an NMD-dependent tumor. In another aspect, the methods comprise preventing an NMD-dependent tumor by administering an effective amount of an NMD-inhibitor described herein to a patient having an NMD-dependent tumor.

In one embodiment, the NMD-inhibitor is an SMG1-inhibitor. In one embodiment, the SMG1-inhibitor is a compound that specifically and/or selectively inhibits the expression of or activity of SMG1. In one embodiment, the SMG1-inhibitor is a compound of formula:

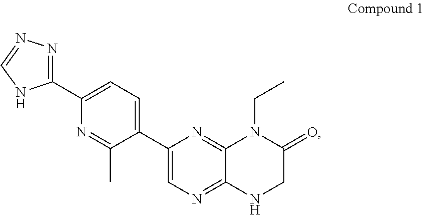 [SMILES: CCN1C(=O)CNc2ncc(-c3ccc(-c4nnc[nH]4)nc3C)nc21]

having the alternative names: 1-ethyl-7-(2-methyl-6-(1H-1,2,4-triazol-3-yl)pyridin-3-yl)-3,4-dihydropyrazino[2,3-b]pyrazin-2(1H)-one; 1-ethyl-7-(2-methyl-6-(4H-1,2,4-triazol-3-yl)pyridin-3-yl)-3,4-dihydropyrazino[2,3-b]pyrazin-2(1H)-one; and 1-ethyl-7-(2-methyl-6-(1H-1,2,4-triazol-5-yl)pyridin-3-yl)-3,4-dihydropyrazino[2,3-b]pyrazin-2(1H)-one, including pharmaceutically acceptable salts, clathrates, hydrates, solvates, stereoisomers, tautomers, metabolites, polymorphs, isotopologues and prodrugs thereof. In one embodiment, the SMG1-inhibitor is Compound 1 or a tautomer or pharmaceutically acceptable salt thereof. In one embodiment, the SMG1-inhibitor is Compound 1 or a tautomer thereof. In one embodiment, the SMG1-inhibitor is Compound 1 or a pharmaceutically acceptable salt thereof. In one embodiment, the SMG1-inhibitor is hydrate or solvate of Compound 1. In one embodiment, the SMG1-inhibitor is a polymorph of Compound 1 including polymorphs of pharmaceutically acceptable salts thereof.

In another aspect is provided a method for treating or preventing an NMD-dependent tumor comprising administering an effective amount of an SMG1-inhibitor to a patient having an NMD-dependent tumor. In another aspect, provided is a method for treating or preventing an SMG1-dependent tumor the method comprising administering an effective amount of an SMG1-inhibitor to a patient having an SMG1-dependent tumor. In another aspect is provided a method for treating an SMG1-dependent tumor comprising administering an effective amount of an SMG1-inhibitor to a patient having an SMG1-dependent tumor. In another aspect is provided a method for preventing an SMG1-dependent tumor comprising administering an effective amount of an SMG1-inhibitor to a patient having an SMG1-dependent tumor. In another aspect is provided a method for treating or preventing an SMG1-dependent tumor comprising administering an effective amount of Compound 1 or a tautomer, solvate, hydrate, co-crystal, clathrate, polymorph, or pharmaceutically acceptable salt thereof to a patient having an SMG1-dependent tumor. In another aspect is provided a method for treating an SMG1-dependent tumor comprising administering an effective amount of Compound 1 or a tautomer, solvate, hydrate, co-crystal, clathrate, polymorph, or pharmaceutically acceptable salt thereof to a patient having an SMG1-dependent tumor. In another aspect is provided a method for preventing an SMG1-dependent tumor comprising administering an effective amount of Compound 1 or a tautomer, solvate, hydrate, co-crystal, clathrate, polymorph, or pharmaceutically acceptable salt thereof to a patient having an SMG1-dependent tumor.

In one embodiment, the SMG1-inhibitor can bind to SMG1. In one embodiment, the binding is covalent. In one embodiment, the binding is non-covalent. In a further embodiment, the SMG1-inhibitor can bind to SMG1 in a cell. In one embodiment, the SMG1-inhibitor is selective for SMG1 by at least 1, 2, 3, 4, 5, 10, 50, 100, or more fold than for another protein (e.g., TORK or DNAPK). In one embodiment, the SMG1-inhibitor is selective for SMG1 by at least 1, 2, 3, 4, 5, 10, 50, 100, or more fold than a PIKK. In one embodiment, the SMG1-inhibitor is selective for SMG1 by at least 1, 2, 3, 4, 5, 10, 50, 100, or more fold than TORK. In one embodiment, the SMG1-inhibitor is specific for SMG1 by at least 1, 2, 3, 4, 5 10, 50, 100, or more fold than for another protein (e.g., TORK or DNAPK). In one embodiment, the SMG1-inhibitor is specific for SMG1 by at least 1, 2, 3, 4, 5 10, 50, 100, or more fold than a PIKK. In one embodiment, the SMG1-inhibitor is specific for SMG1 by at least 1, 2, 3, 4, 5 10, 50, 100, or more fold than TORK.

In one embodiment, the NMD-dependent tumor is a breast tumor, ovarian tumor, papillary thyroid tumor, perivascular epithelioid cell tumor (PEComa), renal tumor, adenocystic carcinoma, chronic lymphocytic leukemia (CLL), myelodysplastic syndrome (MDS), skin melanoma, uveal melanoma, T-ALL, acute myelogenous leukemia (AML), chronic myelomonocytic leukemia (CMML), myelofibrosis, essential thrombocythemia, multiple myeloma (MM), lung tumor, pancreatic tumor, prostate tumor, Wilms tumor, or glioblastoma (e.g. GBM). In one embodiment the NMD-dependent tumor is a solid tumor cancer such as, for example, breast cancer, ovarian cancer, papillary thyroid cancer, perivascular epithelioid cell tumor, renal cancer, adenocystic carcinoma, skin melanoma, uveal melanoma, lung cancer, prostate cancer, pancreatic cancer, Wilm's tumor (nephroblastoma), or glioblastoma. In one embodiment, the NMD-dependent cancer is a hematological cancer such as, for example, CLL, MDS, T-ALL, AML, CMML, myelofibrosis, essential thrombocythemia, or MM. In one embodiment, the NMD-dependent tumor is prostate cancer, for example, castration-resistant prostate cancer. In another embodiment, the NMD-dependent tumor is multiple myeloma. In yet another embodiment, the NMD-dependent tumor is lung cancer, for example, non-small cell lung cancer. In yet another embodiment, the NMD-dependent tumor is myeloproliferative neoplasm (MPN). Accordingly, the methods described herein comprise aspects of treating and preventing the particular NMD-dependent tumors or SMG1-dependent tumors provided herein.

In another aspect is a method of treating and preventing a tumor, where the tumor is CLL, MM, DLBCL, glioblastoma multiforme (GBM), or prostate cancer. In still another aspect is a method of treating a tumor, where the tumor is CLL, MM, DLBCL, glioblastoma multiforme (GBM), or prostate cancer. In yet another aspect is a method of preventing a tumor, where the tumor is CLL, MM, DLBCL, glioblastoma multiforme (GBM), or prostate cancer. In one embodiment, the method comprises treating CLL. In one embodiment, the method comprises treating MM. In one embodiment, the method comprises treating DLBCL. In one embodiment, the method comprises treating GBM. In one embodiment, the method comprises treating prostate cancer. In one embodiment, the method comprises treating NSCLC.

Further provided herein are methods of treating breast cancer, ovarian cancer, papillary thyroid cancer, perivascular epithelioid cell tumor cancer, renal cancer, adenocystic carcinoma, CLL, MDS, skin melanoma, uveal melanoma, T-ALL, AML, CMML, myelofibrosis, essential thrombocythemia, MM, lung cancer, pancreatic cancer, prostate cancer, Wilms tumor (nephroblastoma), or glioblastoma (e.g. GBM) comprising administering an NMD-inhibitor described herein. Further provided herein are methods of treating breast cancer, ovarian cancer, papillary thyroid cancer, perivascular epithelioid cell tumor cancer, renal cancer, adenocystic carcinoma, CLL, MDS, skin melanoma, uveal melanoma, T-ALL, AML, CMML, myeloproliferative neoplasm, myelofibrosis, essential thrombocythemia, MM, lung cancer, pancreatic cancer, prostate cancer, Wilms tumor (nephroblastoma), or glioblastoma (e.g. GBM) comprising administering an NMD-inhibitor described herein. In one embodiment, the NMD-inhibitor is an SMG1-inhibitor. In one embodiment, the SMG1-inhibitor is Compound 1.

Still further provided herein are methods of preventing breast cancer, ovarian cancer, papillary thyroid cancer, perivascular epithelioid cell tumor cancer, renal cancer, adenocystic carcinoma, CLL, MDS, skin melanoma, uveal melanoma, T-ALL, AML, CMML, myelofibrosis, essential thrombocythemia, MM, lung cancer, pancreatic cancer, prostate cancer, Wilms tumor (nephroblastoma), or glioblastoma comprising administering an NMD-inhibitor described herein. Further provided herein are methods of preventing breast cancer, ovarian cancer, papillary thyroid cancer, perivascular epithelioid cell tumor cancer, renal cancer, adenocystic carcinoma, CLL, MDS, skin melanoma, uveal melanoma, T-ALL, AML, CMML, myeloproliferative neoplasm, myelofibrosis, essential thrombocythemia, MM, lung cancer, pancreatic cancer, prostate cancer, Wilms tumor (nephroblastoma), or glioblastoma (e.g. GBM) comprising administering an NMD-inhibitor described herein. In one embodiment, the NMD-inhibitor is an SMG1-inhibitor. In one embodiment, the SMG1-inhibitor is Compound 1.

In certain embodiments, the NMD-dependent tumor is characterized by modulation of the activity or expression of an NMD component described herein. For example, the NMD-dependent tumor is characterized by increased activity or increased expression of an NMD component described herein. In another example, the NMD-dependent tumor is characterized by decreased activity or decreased expression of an NMD component described herein. In certain embodiments, the modulation of activity or expression of the NMD component is a result of a mutation of the mRNA or protein sequence of the NMD component. Provided herein are methods of treating or preventing an NMD-dependent tumor by administering an effective amount of an NMD-inhibitor or an SMG1-inhibitor as described herein for treating an NMD-dependent tumor characterized by modulation of the activity or expression of an NMD component described herein.

In certain embodiments, the NMD component is a protein. In certain embodiments, the NMD component is selected from SMG1, UPF1, UPF2, UPF3b, eRF1, eRF3, SMG5, SMG6, SMG7, SMG8, or SMG9, or a combination thereof. In certain embodiments, the NMD component is SMG1, SMG5, SMG6, SMG7, SMG8, or SMG9, or a combination thereof. In certain embodiments, the NMD component is SMG1.

In certain embodiments, the NMD-dependent tumor is characterized by SMG1 that has modulated activity. For example, SMG1 can have increased activity compared to a control sample of SMG1. In one embodiment, SMG1 can have decreased activity compared to a control sample of SMG1. In certain embodiments, the NMD-dependent tumor is characterized by SMG1 that has a modulated expression level. In certain embodiments, SMG1 can have increased expression compared to a control level of expression of SMG1. In certain embodiments, SMG1 can have decreased expression compared to a control level of expression of SMG1. Control samples can be, for example, a baseline sample taken from a subject prior to beginning any treatment or at a given period of time as described herein. Control levels can be, for example, a baseline level from a sample taken from a subject prior to beginning any treatment or at a given period of time as described herein. Comparison can also, for example, be performed against historical data for a given subject, or population data for similar subjects.

In certain embodiments, the NMD-dependent tumor is characterized by a modulated level of expression of an SMG1-marker. In certain embodiments, the SMG1-marker is selected from 1110004F10Rik, 1110008L16Rik, 1700012D14Rik, 1700037H04Rik, 1810029B16Rik, 2310036022Rik, 2310037I24Rik, 2410004B18Rik, 2900064A13Rik, 4632411B12Rik, 5033414D02Rik, 9030025P20Rik, Aadac, AI314976, AI506816, Abhd12, Acin1, Acot8, Acox1, Actn4, Actr1a, Adss, Aes, Aga, Ahcyl1, Ak2, Akap8, Alg1, Alkbh1, Alkbh3, Alkbh6, Alpk1, Ankfy1, Ankmy1, Ap1b1, Ap2m1, Apobec1, Apoe, Aprt, Arfrp1, Arih2, Arpc1b, Arhgap11b, Ascc2, Atf4, Atf7, Atg2a, Atg3, Atp5j2, Atp6v0d1, Atp6v0e, Atp6v1c1, Atp6v1e1, Aup1, Azin1, BC031181, Bap1, Bcas3, Brd2, Brf2, Bod11, Brp441, Btbd1, Bub3, C2cd3, C6orf141, C6orf48, Ca5b, Cacnb1, Cald1, Camta2, Caprin1, Capns1, Cars2, Cbs, Ccdc111Ccdc130, Ccdc21, Ccnt2, Ccnb1ip1, Cdc3711, Cdk2ap2, Cdkn2aip, Cfi, Cenpa, Cep250, Cept1, Cfb, Cf12, Chkb, Cinp, Clcn4-2, Clcn6, Clcn7, Cldn15, Clk1, Clock, Clptm1, Clta, Cops3, Cops5, Cops7b, Cox7c, Cpsf7, Creg1, Crlf2, Csnk1e, Csnk1g2, Ctage5, Ctla2a, Ctsa, Ctsd, Cxcl16, D10Jhu81e, D4Wsu53e, D6Wsu116e, Dazap1, Dci, Dclk2, Dclre1c, Ddit3, Ddx39, Ddx5, Dhrs3, Dhx15, Dis3, Dnajc25, Dnajc5, Dnajc7, Dnm2, Dpp7, Dpp8, Dscr3, Dtnbp1, Dusp11, Dusp12, Edc4, Edf1, Eef1a1, Eef1b2, Eef2, Eif3k, Eif4a2, Eif4g2, Ell2, Eno1, Epn1, Eprs, Eps1511, Erap2, Ergic1, Eri3, Erp44, Ewsr1, Exosc5, Fam13b, Fam105b, Fam125a, Fam149b, Fam45a, Fbxo18, Fis1, Flot1, Flot2, Foxk2, Fsd11, Fus, Fyb, Gadd45b, Gdf15, Gm15427, Gm3258, Gnl2, Gns, Golga4, Got1, Gp49a, Gpi1, Gramd1a, Grem1, Gm, Gsta3, Gtf2a2, H13, H19, H2-M3, H2-T22, H2afy, Hdac10, Hdgf, Haaol, Hexa, Hira, Hnmpa1, Hnrnpc, Hnrnpd, Hnmph1, Hnmph3, Hnrnpk, Hnrnp1, Hnrp11, Hpca11, Hpn, Hps5, Hras1s, Hsf1, Hspa9, Hyou1, Idua, Ifi16, Ifi203, Ifrd1, Il4ra, Il24, Ilk, Ilkap, Irak1, Irf3, Itfg3, Itsn1, Jak1, Jmjd6, Kars, Khdrbs1, Krcc1, Lamp2, Lars, Lass2, Lats2, Ldlr, Lipg, Lgals1, Loh12cr1, Lpcat, Lsm6, Luc71, Ly6a, Map2k2, Mapkapk3, Matr3, Max, Mbd2, Mbnl1, Mbtps1, Mcm7, Mdm2, Mecr, Mettl21d, Mff, Mgea5, Mina, Mkln1, Mocs, Mocs1, Morf411, Mpdu1, Mpv1712, Mrpl13, Mrps10, Mrps18b, Mta1, Mtpap, Mtx1, Myolf, Naga, Napa, Napb, Nbpf14, Nbr1, Ncaph2, Ndufa7, Ndufa11, Ndufv3, Ndufb5, Ndufs6, Necap2, Nelf, Neu1, Ngf, Nisch, Nkiras1, Nktr, Nom1, Nosip, Nr1h3, Nrbp1, Nt5c2, Nt5c31, Nub1, Nubp2, Nudt9, Nup35, Nxt2, Ociad1, Ogfr, Pabpc4, Pam, Pan3, Papss2, Parp10, Parp6, Pcbp2, Pcmt1, Pcnx12, Pcsk7, Pctk3, Pcyt2, Pddc1, Pecam1, Peci, Pfdn5, Pfldb2, Phc3, Phf1, Phkg2, Pik3ap1, Pilrb1, Pion, Pkm2, Plekha1, Plxnb2, Pnpla7, Ppid, Ppie, Ppp1ca, Ppp1r15a, Ppp4r1, Preb, Prpf38b, Prpf40a, Prpf6, Prr3, Psma3, Psmg2, Ptbp1, Ptpb2, Ptges2, Pttg1ip, R3hdm1, Rab11a, Rab4a, Rabggtb, Rad52, Rassf1, Rbm25, Rbm3, Rbm6, Rbm7, Rce1, Rft1, Rfxank, Ripk1, Rnaseh2a, Rnaset2a, Rnd1, Rnf13, Rnf114, Rnf130, Rnf149, Rnf181, Rnps1, Rpl10a, Rpl12, Rpl13a, Rp11-706o15.1, Rpl17, Rpl3, Rpl37, Rps10, Rps12, Rps19, Rps19bp1, Rps3, Rps6, Rps6kc1, Rps9, Rpsa, Rufy2, Safb2, Sat1, Sdhaf2, Sec11c, Sec61a2, Secisbp2, Senp5, Setd3, Setd4, Setd5, Sidt1, Sf1, Sf3b1, Sf3b3, Srsf11, Srsf2, Srsf3, Srsf4, Srsf5, Srsf6, Srsf8, Srsf9, Sgms1, Sh2b3, Shmt2, Slc15a4, Slc25a12, Slc26a6, Slc40a1, Slc4a2, Slc7a6os, Slc9a8, Smcr71, Smek1, Smg5, Smndc1, Smox, Snrpa1, Snx4, Son, Spns1, Srsf2, Srrm1, Srrt, Stard3, Stom12, Stradb, Stx1a, Supv311, Surf1, Surf2, Tank, Tatdn1, Tbrg4, Tcirg1, Tecr, Tfip11, Thoc3, Thyn1, Tk2, Tm9sf4, Tmbim4, Tmbim6, Tmem11, Tmem149, Tmem156, Tmem183a, Tmem208, Tmem214, Tmem260, Tmem47, Tmem57, Tmem8, Tmem87a, Toe1, Tom112, Tomm34, Top3b, Tpcn1, Tpi1, Tpm1, Tpp2, Tpra1, Tprg1, Tpst2, Tra2a, Tra2b, Trmt1, Trmt6, Trpc1, Trub2, Tsc22d3, Tsg101, Tsku, Ttc33, Ttc39c, Tufm, Tug1, Txnrd1, Tyw5, U2af1, U2af2, Uap111, Ubap2, Ube2d3, Ube2k, Ube2n, Ub15, Ufc1, Ufsp2, Uggt1, phospho-Upf1, Upf2, Use1, Usp7, Vti1b, Vkorc111, Vps11, Vps29, Vps41, Wdfy2, Wdr451, Wdr82, Wdyhv1, Whamm, Xdh, Zc3h7a, Zcchc6, Zcrb1, Zdhhc16, Zdhhc3, Zfand2a, Zfand5, Zfp326, Zfp384, Zfp385a, Zfp523, Zfp553, Zfp655, Zfyve19, Zmat5, Znhit1, Zrsr2, or Zyx, or a combination thereof.

In certain embodiments, the SMG1-marker is selected from 2 or more of the above SMG1-markers. In certain embodiments, the SMG1-marker is selected from 3 or more of the above SMG1-markers. In certain embodiments, the SMG1-marker is selected from 4 or more of the above SMG1-markers. In certain embodiments, the SMG1-marker is selected from 5 or more of the above SMG1-markers. In certain embodiments, the SMG1-marker is selected from 6 or more of the above SMG1-markers. In certain embodiments, the SMG1-marker is selected from 7 or more of the above SMG1-markers. In certain embodiments, the SMG1-marker is selected from 8 or more of the above SMG1-markers. In certain embodiments, the SMG1-marker is selected from 9 or more of the above SMG1-markers. In certain embodiments, the SMG1-marker is selected from 10 or more of the above SMG1-markers. In certain embodiments, the SMG1-marker is selected from 15, 17, 20, 22, 25, 27, 30 or more of the above SMG1-markers. In certain embodiments, the SMG1-marker is selected from each of the above SMG1-markers.

In certain embodiments, the SMG1-marker is selected from phospho-Upf1, Luc71, Neu1, Srsf3, Srsf6, Hnrnp1, Hnrnpk, Rps12, Sf1, U2af1, or Zrsr2 or any combination thereof. In another embodiment, the SMG1-marker is selected from phospho-Upf1, Neu1, Srsf3, Srsf6, Hnrnp1, Hnrnpk, Rps12, Sf1, Luc71, Sf3b1, or Srsf2, U2af1, Zrsr2 or any combination thereof. In still another embodiment, the SMG1-marker is selected from phospho-Upf1, Neu1, Srsf3, Srsf6, Hnrnp1, Hnrnpk, Rps12, or Sf1, Sf3b1, U2af1, Zrsr2 or any combination thereof. In other embodiments, the SMG1-marker is selected from Srsf3, Hnrnp1, Rps12, Hnrnpk, Neu1, or Sf1, or any combination thereof. In another embodiment, the SMG1-markers is selected from 2, 3, 4, 5, 6, 7, 8 or all of phospho-UPF1, Luc71, Neu1, Srsf3, Srsf6, Hnrnp1, Hnrnp1, Rps12, or Sf1, Sf3b1, U2af1, Zrsr2 or any combination thereof. In another embodiment, the SMG1-markers is selected from 2, 3, 4, 5, 6, 7, or all of phospho-Upf1, Neu1, Srsf3, Srsf6, Hnrnp1, Hnrnpk, Rps12, or Sf1, Sf3b1, U2af1, Zrsr2 or any combination thereof. In some such embodiments, the NMD-dependent tumor is prostate cancer, for example, castration-resistant prostate cancer. In another embodiment, the NMD-dependent tumor is multiple myeloma. In yet another embodiment, the NMD-dependent tumor is lung cancer, for example, non-small cell lung cancer.

In certain embodiments, the NMD-dependent tumor is characterized by dysregulated splicing of one or more cancer-associated genes. In one embodiment, the cancer associated gene is selected from AIMP2, AR, BCL-X, BCRL11, BIRCS, BRAF, BRD4, CASP3, CD22, CD44, CDH17, CEACAM1, CLDN18, CPE, CXCL12, EGFR, ENHA, FAS, FGFR2, FN1, FPGS, HIF1A, IKZF1, KLF6, KLK8, MCL1, MDM2, MET, MKNK2, REST4, S6K1, SYK, TERT, TP53, VEGFA, or WFDC2, or a combination thereof. In one embodiment, the cancer associated gene is selected from 2, 3, 4, 5, 6, 7, 8, 9, 10 or more of AIMP2, AR, BCL-X, BCRL11, BIRCS, BRAF, BRD4, CASP3, CD22, CD44, CDH17, CEACAM1, CLDN18, CPE, CXCL12, EGFR, ENHA, FAS, FGFR2, FN1, FPGS, HIF1A, IKZF1, KLF6, KLK8, MCL1, MDM2, MET, MKNK2, REST4, S6K1, SYK, TERT, TP53, VEGFA, or WFDC2. In one embodiment, the cancer associated gene is selected from 15, 18, 20, 22, 25, 30 or more of AIMP2, AR, BCL-X, BCRL11, BIRCS, BRAF, BRD4, CASP3, CD22, CD44, CDH17, CEACAM1, CLDN18, CPE, CXCL12, EGFR, ENHA, FAS, FGFR2, FN1, FPGS, HIF1A, IKZF1, KLF6, KLK8, MCL1, MDM2, MET, MKNK2, REST4, S6K1, SYK, TERT, TP53, VEGFA, or WFDC2. In one embodiment, the cancer associated gene is selected from one or more of AIMP2, AR, BCL-X, BCRL11, BIRC5, BRAF, BRD4, CASP3, CD22, CD44, CDH17, CEACAM1, CLDN18, CPE, CXCL12, EGFR, ENHA, FAS, FGFR2, FN1, FPGS, HIF1A, IKZF1, KLF6, KLK8, MCL1, MDM2, MET, MKNK2, REST4, S6K1, SYK, TERT, TP53, VEGFA, or WFDC2, or a combination thereof. In another embodiment, the cancer associated gene is selected from one or more of WT1, RB, BRCA1, BRCA2, MRE11, E-cadherin, MLH1, EP300, CHK2, EPHB2, JAK1, MIK67, YPLM1, USP9X, CSMD3, MGA, ASXL2, ZNF292, MAP3K1, or ZFHX3, or any combination thereof. In one embodiment, the cancer associated gene is selected from 2, 3, 5, 7, 10, 12, 15, 18 or more of WT1, RB, BRCA1, BRCA2, MRE11, E-cadherin, MLH1, EP300, CHK2, EPHB2, JAK1, MIK67, YPLM1, USP9X, CSMD3, MGA, ASXL2, ZNF292, MAP3K1, or ZFHX3, or any combination thereof.

Provided herein are methods of treating or preventing an NMD-dependent tumor (or an SMG1-dependent tumor) by administering an effective amount of an NMD-inhibitor or an SMG1-inhibitor as described herein for treating an NMD-dependent tumor characterized by dysregulated splicing of one or more cancer-associated genes.

In one embodiment, the cancer associated gene is selected from one or more of BCL-X, BRAF, BRD3, CASP3, CD44, EGFR, FAS, HIF1A, MCL1, MDM2, VEGF, AR, TP53, WT1, RB, BRCA1, BRCA2, MRE11, E-cadherin, MLH1, EP300, CHK2, EPHB2, JAK1, MIK67, YPLM1, USP9X, CSMD3, MGA, ASXL2, ZNF292, MAP3K1, ZFHX3, or any combination thereof. In one embodiment, the cancer associated gene is selected from 2, 3, 5, 7, 10, 15, 20, 25 or more of BCL-X, BRAF, BRD3, CASP3, CD44, EGFR, FAS, HIF1A, MCL1, MDM2, VEGF, AR, TP53, WT1, RB, BRCA1, BRCA2, MRE11, E-cadherin, MLH1, EP300, CHK2, EPHB2, JAK1, MIK67, YPLM1, USP9X, CSMD3, MGA, ASXL2, ZNF292, MAP3K1, or ZFHX3.

In one embodiment, the cancer associated gene is selected from one or more of BCL-X, BRAF, BRD3, CASP3, CD44, EGFR, FAS, HIF1A, MCL1, MDM2, VEGF, AR, TP53, WT1, RB, BRCA1, BRCA2, or any combination thereof. In one embodiment, the cancer associated gene is selected from 2, 3, 5, 7, 10, 15, or more of BCL-X, BRAF, BRD3, CASP3, CD44, EGFR, FAS, HIF1A, MCL1, MDM2, VEGF, AR, TP53, WT1, RB, BRCA1, or BRCA2.

In one embodiment, the cancer associated gene is selected from one or more of BCL-X, BRAF, BRD3, CASP3, CD44, EGFR, FAS, HIF1A, MCL1, MDM2, VEGF, AR, TP53, WT1, RB, BRCA1, BRCA2, MLH1, EP300, CHK2, EPHB2, JAK1, or any combination thereof. In one embodiment, the cancer associated gene is selected from 2, 3, 5, 7, 10, 15, or more of BCL-X, BRAF, BRD3, CASP3, CD44, EGFR, FAS, HIF1A, MCL1, MDM2, VEGF, AR, TP53, WT1, RB, BRCA1, BRCA2, MLH1, EP300, CHK2, EPHB2, or JAK1.

In certain embodiments, the NMD-dependent tumor is characterized by dysregulated splicing of one or more cancer-associated genes selected from one or more of BCL-X, BRAF, BRD3, CASP3, CD44, EGFR, FAS, HIF1A, MCL1, MDM2, VEGF, AR, TP53, WT1, RB, BRCA1, or BRCA2, and one or more of MRE11, E-cadherin, MLH1, EP300, CHK2, EPHB2, or JAK1. In certain embodiments, the NMD-dependent tumor is characterized by dysregulated splicing of one or more cancer-associated genes selected from one or more of BCL-X, BRAF, BRD3, CASP3, CD44, EGFR, FAS, HIF1A, MCL1, MDM2, VEGF, AR, TP53, WT1, RB, BRCA1, or BRCA2, and one or more of MIK67, YPLM1, USP9X, CSMD3, MGA, ASXL2, ZNF292, MAP3K1, or ZFHX3. In certain embodiments, the NMD-dependent tumor is characterized by dysregulated splicing of one or more cancer-associated genes selected from one or more of BCL-X, BRAF, BRD3, CASP3, CD44, EGFR, FAS, HIF1A, MCL1, MDM2, VEGF, AR, TP53, WT1, RB, BRCA1, or BRCA2, and one or more of MRE11, E-cadherin, MLH1, EP300, CHK2, EPHB2, JAK1, MIK67, YPLM1, USP9X, CSMD3, MGA, ASXL2, ZNF292, MAP3K1, or ZFHX3.

In certain embodiments, the NMD-dependent tumor is characterized by modulation of the activity or expression of one or more splicing factors. In one embodiment, the NMD-dependent tumor is characterized by modulation of the activity of one or more splicing factors. For example, in one embodiment, the NMD-dependent tumor is characterized by increased activity of one or more splicing factors compared to a control level of activity. In one embodiment, the NMD-dependent tumor is characterized by decreased activity of one or more splicing factors compared to a control level of activity. In another embodiment, the NMD-dependent tumor is characterized by the modulation of the expression of one or more splicing factors. For example, in one embodiment, the NMD-dependent tumor is characterized by increased levels of expression compared to a control level of expression. In one embodiment, the NMD-dependent tumor is characterized by decreased levels of expression compared to a control level of expression. In certain embodiments, the modulation of the activity or expression of the one or more splicing factors is a result of a mutation of the nucleotide or protein sequence of the splicing factor. Provided herein are methods of treating or preventing an NMD-dependent tumor (or an SMG1-dependent tumor) by administering an effective amount of an NMD-inhibitor or an SMG1-inhibitor as described herein for treating an NMD-dependent tumor characterized by modulation of the activity or expression of one or more splicing factors.

In certain embodiments, the splicing factor is selected from DDX23, DDX46, DHX16, DHX38, DHX8, DIMT1, EFTUD2, HNRNPF, HNRNPH1, HNRNPL, LUC7L2, LUC7L3, PRPF19, PRPF3, PRPF31, PRPF39, PRPF4, PRPF40A, PRPF6, PRPF8, SF1, SF3A1, SF3A2, SF3A3, SF3B1, SF3B14, SF3B2, SF3B3, SF3B5, SNRNP200, SNRNP35, SNRNP40, SNRNP70, SNRPB, SNRPC, SNRPD1, SNRPD2, SNRPD3, SNRPE, SNRPF, SNRPG, SRSF1, SRSF10, SRSF2, SRSF3, SRSF5, SRSF6, SRSF7, U2AF1, U2AF2, ZRSR2, AAR2, ACIN1, ALYREF, AQR, BCAS2, BUB3, BUD13, BUD31, C1ORF55, C90RF78, CACTIN, CCNDBP1, CD2BP2, CDC40, CDC5L, CDK11A, CHERP, CRNKL1, CWC25, CXORF56, DDX35, DDX39B, DDX41, DDX5, DGCR14, DHX15, DNAJC8, EIF4A3, FAM32A, FAM50A, FAM50B, FRA1OAC1, FRG1, FUS, HSP27, HSP73, ISY1, MAGOH, MAGOH, MFAP1, NCBP2, NHP2L1, NOSIP, PLRG1, PPIG, PPIH, PPIL1, PPWD1, PRL1, PRPF18, PRPF38B, PUF60, RAVER1, RBM10, RBM14, RBM17, RBM22, RBM25, RBM4, RBM8A, RBMX2, RMB5, SART1, SF3B4, SF3B6, SKIV2L2, SLUT, SMNDC1, SMU1, SNRPB2, SNU13, SNW1, SRRM2, SUGP1, TCERG1, THOC1, THOC2, THOC3, TXNL4A, U2SURP, UBL5, WBP11, WDR83, XAB2, XAB2, ZMAT2, or ZNF830, or a combination thereof.

In certain embodiments, the splicing factor is selected from 2 or more of the above splicing factors. In certain embodiments, the splicing factor is selected from 3 or more of the above splicing factors. In certain embodiments, the splicing factor is selected from 4 or more of the above splicing factors. In certain embodiments, the splicing factor is selected from 5 or more of the above splicing factors. In certain embodiments, the splicing factor is selected from 6 or more of the above splicing factors. In certain embodiments, the splicing factor is selected from 7 or more of the above splicing factors. In certain embodiments, the splicing factor is selected from 8 or more of the above splicing factors. In certain embodiments, the splicing factor is selected from 9 or more of the above splicing factors. In certain embodiments, the splicing factor is selected from 10 or more of the above splicing factors. In certain embodiments, the splicing factor is selected from 15, 17, 20, 22, 25, 27, 30 or more of the above splicing factors. In certain embodiments, the splicing factor is selected from each of the above splicing factors.

In certain embodiments, the splicing factor is selected from DDX23, DDX46, DHX16, DHX38, DHX8, DIMT1, EFTUD2, HNRNPF, HNRNPH1, HNRNPL, LUC7L2, LUC7L3, PRPF19, PRPF3, PRPF31, PRPF39, PRPF4, PRPF40A, PRPF6, PRPF8, SF1, SF3A1, SF3A2, SF3A3, SF3B1, SF3B14, SF3B2, SF3B3, SF3B5, SNRNP200, SNRNP35, SNRNP40, SNRNP70, SNRPB, SNRPC, SNRPD1, SNRPD2, SNRPD3, SNRPE, SNRPF, SNRPG, SRSF1, SRSF10, SRSF2, SRSF3, SRSF5, SRSF6, SRSF7, U2AF1, U2AF2, ZRSR2, or a combination thereof.

In certain embodiments, the splicing factor is selected from 2, 3, 4, 5, 6, 7, 8, 9, 10, or more of DDX23, DDX46, DHX16, DHX38, DHX8, DIMT1, EFTUD2, HNRNPF, HNRNPH1, HNRNPL, LUC7L2, LUC7L3, PRPF19, PRPF3, PRPF31, PRPF39, PRPF4, PRPF40A, PRPF6, PRPF8, SF1, SF3A1, SF3A2, SF3A3, SF3B1, SF3B14, SF3B2, SF3B3, SF3B5, SNRNP200, SNRNP35, SNRNP40, SNRNP70, SNRPB, SNRPC, SNRPD1, SNRPD2, SNRPD3, SNRPE, SNRPF, SNRPG, SRSF1, SRSF10, SRSF2, SRSF3, SRSF5, SRSF6, SRSF7, U2AF1, U2AF2, or ZRSR2.

In certain embodiments, the splicing factor is selected from 12, 15, 18, 20, 22, 25, 30, or more of DDX23, DDX46, DHX16, DHX38, DHX8, DIMT1, EFTUD2, HNRNPF, HNRNPH1, HNRNPL, LUC7L2, LUC7L3, PRPF19, PRPF3, PRPF31, PRPF39, PRPF4, PRPF40A, PRPF6, PRPF8, SF1, SF3A1, SF3A2, SF3A3, SF3B1, SF3B14, SF3B2, SF3B3, SF3B5, SNRNP200, SNRNP35, SNRNP40, SNRNP70, SNRPB, SNRPC, SNRPD1, SNRPD2, SNRPD3, SNRPE, SNRPF, SNRPG, SRSF1, SRSF10, SRSF2, SRSF3, SRSF5, SRSF6, SRSF7, U2AF1, U2AF2, or ZRSR2. In one embodiment, the splicing factor is selected from all of DDX23, DDX46, DHX16, DHX38, DHX8, DIMT1, EFTUD2, HNRNPF, HNRNPH1, HNRNPL, LUC7L2, LUC7L3, PRPF19, PRPF3, PRPF31, PRPF39, PRPF4, PRPF40A, PRPF6, PRPF8, SF1, SF3A1, SF3A2, SF3A3, SF3B1, SF3B14, SF3B2, SF3B3, SF3B5, SNRNP200, SNRNP35, SNRNP40, SNRNP70, SNRPB, SNRPC, SNRPD1, SNRPD2, SNRPD3, SNRPE, SNRPF, SNRPG, SRSF1, SRSF10, SRSF2, SRSF3, SRSF5, SRSF6, SRSF7, U2AF1, U2AF2, or ZRSR2.

In certain embodiments, the splicing factor is selected from SF1, SF3A1, SF3A2, SF3A3, SF3B1, SF3B14, SF3B2, SF3B3, SF3B5, SRSF1, SRSF10, SRSF2, SRSF3, SRSF5, SRSF6, SRSF7, U2AF1, U2AF2, ZRSR2, or any combination thereof. In one embodiment, the splicing factor is selected from two or more of SF1, SF3A1, SF3A2, SF3A3, SF3B1, SF3B14, SF3B2, SF3B3, SF3B5, SRSF1, SRSF10, SRSF2, SRSF3, SRSF5, SRSF6, SRSF7, U2AF1, U2AF2, or ZRSR2. In another embodiment, the splicing factor is selected from 1, 2, 3, 5, 8, 10, 12, 15, or more of SF1, SF3A1, SF3A2, SF3A3, SF3B1, SF3B14, SF3B2, SF3B3, SF3B5, SRSF1, SRSF10, SRSF2, SRSF3, SRSF5, SRSF6, SRSF7, U2AF1, U2AF2, or ZRSR2. In still another embodiment, the splicing factor is selected from all of SF1, SF3A1, SF3A2, SF3A3, SF3B1, SF3B14, SF3B2, SF3B3, SF3B5, SRSF1, SRSF10, SRSF2, SRSF3, SRSF5, SRSF6, SRSF7, U2AF1, U2AF2, and ZRSR2.

In certain embodiments, the NMD-dependent tumor is characterized by dysregulated splicing due to modulation of the activity or expression of one or more splicing regulators. In one embodiment, the NMD-dependent tumor is characterized by modulation of the activity of one or more splicing regulators. For example, in one embodiment, the NMD-dependent tumor is characterized by increased activity of one or more splicing regulators compared to a control level of activity. In one embodiment, the NMD-dependent tumor is characterized by decreased activity of one or more splicing regulators compared to a control level of activity. In another embodiment, the NMD-dependent tumor is characterized by the modulation of the expression of one or more splicing regulators. For example, in one embodiment, the NMD-dependent tumor is characterized by increased levels of expression compared to a control level of expression. In one embodiment, the NMD-dependent tumor is characterized by decreased levels of expression compared to a control level of expression. In certain embodiments, the modulation of the activity or expression of the one or more splicing regulators is a result of a mutation of the sequence of the splicing regulator. In certain embodiments, the one or more splicing regulators comprise a mutation. Provided herein are methods of treating or preventing an NMD-dependent tumor (or an SMG1-dependent tumor) by administering an effective amount of an NMD-inhibitor or an SMG1-inhibitor as described herein for treating an NMD-dependent tumor characterized by dysregulated splicing due to modulation of the activity or expression of one or more splicing regulators.

In certain embodiments, the splicing regulator is selected from SSFPQ, AFF2, CASC3, CCAR1, CDK12, CELF2, SNRPB, DDX5, DDX39A, DHX9, DHX15, DHX35, GEMIN6, HNRNPC, HNRNPH, HSPA8, MBNL1, NONO, PABPC1, PCBP1, PPIH, PRMT7, PPP2R1A, PRPF28B, RBFOX2, RHEB, PSIP1, PRPF4, RBM10, RBMX, SMC1A, SMNDC1, SNRPA, SNRPB, SNRPD1, SNRPD2, SNRPF, SNRPN, SON, SRRM2, SCNM1, SRRM2, SYNCRIP, TXNL4B, U2AF35, WBP11, or WT1, or any combination thereof.

In certain embodiments, the splicing regulator is selected from 1, 2, 3, 4, 5, 6, 7, 8, 9, 10, or more of SSFPQ, AFF2, CASC3, CCAR1, CDK12, CELF2, SNRPB, DDX5, DDX39A, DHX9, DHX15, DHX35, GEMIN6, HNRNPC, HNRNPH, HSPA8, MBNL1, NONO, PABPC1, PCBP1, PPIH, PRMT7, PPP2R1A, PRPF28B, RBFOX2, RHEB, PSIP1, PRPF4, RBM10, RBMX, SMC1A, SMNDC1, SNRPA, SNRPB, SNRPD1, SNRPD2, SNRPF, SNRPN, SON, SRRM2, SCNM1, SRRM2, SYNCRIP, TXNL4B, U2AF35, WBP11, or WT1. In certain embodiments, the splicing regulator is selected from 12, 23, 15, 18, 20, 25, 30, 35 or more of SSFPQ, AFF2, CASC3, CCAR1, CDK12, CELF2, SNRPB, DDX5, DDX39A, DHX9, DHX15, DHX35, GEMIN6, HNRNPC, HNRNPH, HSPA8, MBNL1, NONO, PABPC1, PCBP1, PPIH, PRMT7, PPP2R1A, PRPF28B, RBFOX2, RHEB, PSIP1, PRPF4, RBM10, RBMX, SMC1A, SMNDC1, SNRPA, SNRPB, SNRPD1, SNRPD2, SNRPF, SNRPN, SON, SRRM2, SCNM1, SRRM2, SYNCRIP, TXNL4B, U2AF35, WBP11, or WT1. In one embodiment, the splicing regulator is selected from all of SSFPQ, AFF2, CASC3, CCAR1, CDK12, CELF2, SNRPB, DDX5, DDX39A, DHX9, DHX15, DHX35, GEMIN6, HNRNPC, HNRNPH, HSPA8, MBNL1, NONO, PABPC1, PCBP1, PPIH, PRMT7, PPP2R1A, PRPF28B, RBFOX2, RHEB, PSIP1, PRPF4, RBM10, RBMX, SMC1A, SMNDC1, SNRPA, SNRPB, SNRPD1, SNRPD2, SNRPF, SNRPN, SON, SRRM2, SCNM1, SRRM2, SYNCRIP, TXNL4B, U2AF35, WBP11, or WT1, or any combination thereof.

Also provided herein are methods for treating a cancer associated with abnormally greater protein production, such as multiple myeloma, by administering an effective amount of Compound 1 to a patient having multiple myeloma. In another aspect is a method of treating a cancer associated with abnormally greater protein production, such as multiple myeloma, by administering an effective amount of an SMG1-inhibitor described herein to a patient having multiple myeloma. In such embodiments, the cancer is characterized as described herein (e.g., modulated activity or expression of: an NMD-component described herein, SMG1, an SMG1-marker described herein, one or more splicing factors, or one or more splicing regulators; or by dysregulated splicing as described herein).

Provided herein are methods of treating a cancer with dysregulated splicing, such as T-cell lymphoma by administering an effective amount of an SMG1-inhibitor described herein to a patient having multiple myeloma. In another aspect is a method for treating a cancer associated with dysregulated splicing, such as T-cell lymphoma by administering an effective amount of Compound 1 to a patient having multiple myeloma. In such embodiments, the T-cell lymphoma is characterized as described herein (e.g., modulated activity or expression of: an NMD-component described herein, SMG1, an SMG1-marker described herein, one or more splicing factors, or one or more splicing regulators; or by dysregulated splicing as described herein).

In certain embodiments, provided herein are methods of treating an NMD-dependent tumor characterized by a mutation in SRSF2, U2AF1, SF3B1 or ZRSR2, wherein the NMD-dependent tumor is selected from breast cancer, CLL, MDS, uveal melanoma, T-ALL, AML (for example, blastic plasmacytoid dendritic cell neoplasm, MPDCN), CMML, myeloproliferative neoplasm (MPN), pancreatic cancer, or glioblastoma. In other embodiments, provided herein are methods of treating an NMD-dependent tumor characterized by a mutation in BRCA1 or BRCA2, wherein the NMD-dependent tumor is selected from breast cancer, or ovarian cancer. In yet other embodiments, provided herein are methods of treating an NMD-dependent tumor characterized by a mutation in p53, (for example, a p53 mutation which renders the resultant p53 RNA an NMD target), wherein the NMD-dependent tumor is selected from breast cancer, pancreatic cancer, colorectal cancer (CRC), head and neck small cell carcinoma (HNSCC), esophageal cancer, female genital cancer, skin cancer, stomach concern, liver cancer, urinary tract cancer, or ovarian cancer (for example high grade serous carcinoma of the ovary).

The methods described herein can also comprise coadministering a second active agent with the NMD-inhibitor or SMG1-inhibitor described herein. For example, the methods described herein comprise administering to a patient described herein an effective amount of an SMG1-inhibitor as a component of a combination therapy described below. In one embodiment, the methods described herein comprise administering to a patient described herein an effective amount of an NMD-inhibitor or SMG1-inhibitor described herein and one or more of an immuno-oncology treatment, a proteasome inhibitor, a premature termination codon (PTC) read-through compound, an NMD-inhibitor, or a splice factor inhibitor, each as described herein.

In another embodiment, the methods described herein comprise administering an effective amount of an NMD-inhibitor or SMG1-inhibitor as described herein to a patient provided herein and one or more of (i) a CTLA4 inhibitor; (ii) a PD-1 inhibitor; (iii) a PD-L1 inhibitor; (iv) a PD-L2 inhibitor; (v) a LAG3 inhibitor; (vi) an antibody to B7-H3; (vii) a TIM-3 inhibitor; (viii) an OX40 agonist; (ix) a CXCR4 inhibitor; (x) a CSF1R inhibitor; (xi) an ICOS inhibitor; (xii) a Toll-like receptor agonist; (xiii) a 41-BB agonist; (xiv) a GITR agonist; (xv) a CD27 agonist; (xvi) a CD40 agonist; (xvii) an IDO inhibitor; (xviii) an IDO1-derived peptide; or (xix) an IDO inhibitor. In one embodiment the NMD-inhibitor or SMG1-inhibitor is administered according to the methods described herein in combination with a (i) a CTLA4 inhibitor; (ii) a PD-1 inhibitor; (iii) a PD-L1 inhibitor; (iv) a PD-L2 inhibitor; (v) a Toll-like receptor agonist; or (vi) an IDO inhibitor.

Further provided herein are methods of inhibiting SMG1 in vivo by contacting SMG1 with an SMG1-inhibitor. In one embodiment, the SMG1-inhibitor is Compound 1.

Further provided herein are methods of testing for responsiveness to an NMD-inhibitor in a patient. Such methods comprise screening a biological sample obtained from a patient for the presence of an NMD-marker described herein, wherein the presence of the NMD-marker indicates an increased likelihood that the patient will be responsive to treatment with the NMD-inhibitor. In certain embodiments, the NMD-marker is an SMG1-marker as described herein. In certain embodiments, the SMG1-marker is selected from phospho-Upf1, Luc71, Neu1, Srsf3, Srsf6, Hnrnp1, Hnrnpk, Rps12, or Sf1. In certain embodiments, the SMG1-marker is selected from phospho-Upf1, Neu1, Srsf3, Srsf6, Hnrnp1, Hnrnpk, Rps12, Sf1, Luc71, Sf3b1, or Srsf2, U2af1, Zrsr2, or any combination thereof. In still another embodiment, the SMG1-marker is selected from phospho-Upf1, Neu1, Srsf3, Srsf6, Hnrnp1, Hnrnpk, Rps12, or Sf1, U2af1, Zrsr2, or any combination thereof. In one embodiment, the NMD-inhibitor is Compound 1.

The methods of testing for responsiveness to an NMD-inhibitor described herein can be performed before beginning treatment with the NMD-inhibitor. In one embodiment, the methods of testing for responsiveness can be performed during the course of treatment with the NMD-inhibitor. In such embodiments, the presence of the NMD-marker can signify efficacy of the treatment. In another embodiment, the responsiveness to the NMD-inhibitor is tested after administration of a second active agent such as those described herein (e.g., an immuno-oncology treatment, a proteasome inhibitor, a premature termination codon (PTC) read-through compound, an NMD-inhibitor, or a splice factor inhibitor).

Further provided herein are methods of testing for responsiveness to an SMG1-inhibitor for treating or preventing an SMG1-dependent tumor in a patient. Such methods comprise screening a biological sample obtained from a patient for the presence of an SMG1-marker, wherein the presence of the SMG1-marker indicates an increased likelihood that the patient will be responsive to treatment with the SMG1-inhibitor.

The methods of testing for responsiveness to an SMG1-inhibitor described herein can be performed before beginning treatment with the SMG1-inhibitor. In one embodiment, the methods of testing for responsiveness can be performed during the course of treatment with the SMG1-inhibitor. In such embodiments, the presence of the SMG1-marker can signify efficacy of the treatment. In another embodiment, the responsiveness to the SMG1-inhibitor is tested after administration of a second active agent such as those described herein (e.g., an immuno-oncology treatment, a proteasome inhibitor, a premature termination codon (PTC) read-through compound, an NMD-inhibitor, or a splice factor inhibitor).

In another aspect provided herein is a method of testing for responsiveness to an SMG1-inhibitor for treating an SMG1-dependent tumor in a patient by screening a biological sample obtained from a patient for the presence of an SMG1-marker, wherein the presence of the SMG1-marker indicates an increased likelihood that the patient will be responsive to the SMG1-inhibitor. In one embodiment, the SMG1-inhibitor is Compound 1.

In another aspect is a method of testing for responsiveness to an SMG1-inhibitor for preventing an SMG1-dependent tumor in a patient by screening a biological sample obtained from a patient for the presence of an SMG1-marker, wherein the presence of the SMG1-marker indicates an increased likelihood that the patient will be responsive to the SMG1-inhibitor.

Further provided herein are methods for testing for responsiveness to an SMG1-inhibitor administered to a patient for treatment or prevention of an NMD-dependent tumor by: (a) measuring a level of expression or activity of an SMG1-marker in a biological sample obtained from the patient; (b) administering a dosage amount of the SMG1-inhibitor to the patient; (c) measuring a level of expression or activity of the SMG1-marker in a second biological sample obtained from the patient after the administration of the dosage amount; (d) comparing the levels of expression or activity of the SMG1-marker from the first and the second biological samples; (e) wherein a modulation of the level of expression or activity of the SMG1-marker indicates an increased likelihood that the patient will be responsive to treatment with the SMG1-inhibitor. In certain embodiments, the modulation is an increase in the level of expression of the SMG1-marker. In certain embodiments, the modulation is an increase in the activity of the SMG1-marker. In certain embodiments, the modulation is a decrease in the level of expression of the SMG1-marker. In certain embodiments, the modulation is a decrease in the activity of the SMG1-marker. The dosage amount is a dosage amount provided herein. The dosage amount can comprise an effective amount of the SMG1-inhibitor. In one embodiment, the dosage amount comprises an effective amount of Compound 1. In one embodiment, step (a) comprises measuring a level of expression or activity of at least 2, 3, 4, 5, 6, 7, 8, 9, or 10 SMG1-markers described herein.

Such methods can be performed before beginning treatment with the SMG1-inhibitor. In one embodiment, the methods can be performed during the course of treatment with the SMG1-inhibitor. In such embodiments, the presence of the SMG1-marker can signify efficacy of the treatment. In another embodiment, the responsiveness to the SMG1-inhibitor is tested after administration of a second active agent such as those described herein (e.g., an immuno-oncology treatment, a proteasome inhibitor, a premature termination codon (PTC) read-through compound, an NMD-inhibitor, or a splice factor inhibitor).

In certain embodiments, the SMG1-marker of the methods described herein comprises one or more of phospho-UPF1, LUC7L, NEU1, SRSF3, SRSF6, HNRNPL, HNRNPK, RPS12, or SF1, or any combination thereof. In certain embodiments, the SMG1-marker of the methods described herein comprises two or more of phospho-UPF1, LUC7L, NEU1, SRSF3, SRSF6, HNRNPL, HNRNPK, RPS12, or SF1. In certain embodiments, the SMG1-marker of the methods described herein comprises at least 3, 4, 5, or all of phospho-UPF1, LUC7L, NEU1, SRSF3, SRSF6, HNRNPL, HNRNPK, RPS12, or SF1. In one embodiment, the method includes measuring each of phospho-UPF1, LUC7L, NEU1, SRSF3, SRSF6, HNRNPL, HNRNPK, RPS12, SF1, U2af1, or Zrsr2. In another embodiment, the SMG1-marker comprises at least SF3B1. In another embodiment, the SMG1-marker comprises at least phospho-Upf1. In certain embodiments, the SMG1-marker of the methods described herein comprises phospho-Upf1, Neu1, Srsf3, Srsf6, Hnrnp1, Hnrnpk, Rps12, Sf1, Luc71, Sf3b1, or Srsf2, or any combination thereof. In certain embodiments, the SMG1-marker of the methods described herein comprises 2, 3, 4, 5, 6, 7, 8, 9, 10, or all of phospho-Upf1, Neu1, Srsf3, Srsf6, Hnrnp1, Hnrnpk, Rps12, Sf1, Luc71, Sf3b1, Srsf2, U2af1, or Zrsr2. In certain embodiments, the SMG1-marker of the methods described herein comprises phospho-Upf1, Neu1, Srsf3, Srsf6, Hnrnp1, Hnrnpk, Rps12, or Sf1, or any combination thereof. In certain embodiments, the SMG1-marker of the methods described herein comprises 2, 3, 4, 5, 6, 7, or all of phospho-Upf1, Neu1, Srsf3, Srsf6, Hnrnp1, Hnrnpk, Rps12, or Sf1, or any combination thereof.

The methods above can further comprise testing for a mutation of one or more tumor suppressor genes. In certain embodiments, the presence of a mutation to one or more tumor suppressor genes can indicate the patient will be responsive to treatment with an SMG1-inhibitor described herein (e.g., Compound 1). Exemplary tumor suppressor genes (e.g. cancer associated genes) include, but are not limited to, p53, WT1, RB, BRCA1, BRCA2, MRE11, E-cadherin, MLH1, EP300, CHK2, EPHB2, JAK1, MIK67, YPLM1, USP9X, CSMD3, MGA, ASXL2, ZNF292, MAP3K1, and ZFHX3, or any combination thereof. In one embodiment, the tumor suppressor genes comprise p53, WT1, RB, BRCA1, or BRCA2, or any combination thereof. In one embodiment, the tumor suppressor genes comprise MRE11, E-cadherin, MLH1, EP300, CHK2, EPHB2, or JAK1, or any combination thereof. In another embodiment, the tumor suppressor genes comprise MIK67, YPLM1, USP9X, CSMD3, MGA, ASXL2, ZNF292, MAP3K1, or ZFHX3, or any combination thereof. In certain embodiments, the tumor suppressor genes comprise one or more of p53, WT1, RB, BRCA1, or BRCA2 and one or more of MRE11, E-cadherin, MLH1, EP300, CHK2, EPHB2, JAK1, MIK67, YPLM1, USP9X, CSMD3, MGA, ASXL2, ZNF292, MAP3K1, or ZFHX3.

Provided herein are methods for testing for responsiveness to an SMG1-inhibitor administered to a patient for treatment of multiple myeloma by: (a) measuring a level of expression or activity of an SMG1-marker in a biological sample obtained from the patient; (b) administering a dosage amount of the SMG1-inhibitor to the patient; (c) measuring a level of expression or activity of the SMG1-marker in a second biological sample obtained from the patient after the administration of the dosage amount; (d) comparing the levels of expression or activity of the SMG1-marker from the first and the second biological samples; (e) wherein a modulation of the level of expression or activity of the SMG1-marker indicates an increased likelihood that the patient will be responsive to treatment with the SMG1-inhibitor.

Provided herein are methods for testing for responsiveness to an SMG1-inhibitor administered to a patient for treatment of acute myelogenous leukemia by: (a) measuring a level of expression or activity of an SMG1-marker in a biological sample obtained from the patient; (b) administering a dosage amount of the SMG1-inhibitor to the patient; (c) measuring a level of expression or activity of the SMG1-marker in a second biological sample obtained from the patient after the administration of the dosage amount; (d) comparing the levels of expression or activity of the SMG1-marker from the first and the second biological samples; (e) wherein a modulation of the level of expression or activity of the SMG1-marker indicates an increased likelihood that the patient will be responsive to treatment with the SMG1-inhibitor.

Provided herein are methods for testing for responsiveness to an SMG1-inhibitor administered to a patient for treatment of a cancer associated with dysregulated splicing, such as T-cell lymphoma by: (a) measuring a level of expression or activity of an SMG1-marker in a biological sample obtained from the patient; (b) administering a dosage amount of the SMG1-inhibitor to the patient; (c) measuring a level of expression or activity of the SMG1-marker in a second biological sample obtained from the patient after the administration of the dosage amount; (d) comparing the levels of expression or activity of the SMG1-marker from the first and the second biological samples; (e) wherein a modulation of the level of expression or activity of the SMG1-marker indicates an increased likelihood that the patient will be responsive to treatment with the SMG1-inhibitor. In one embodiment, step (a) of the methods provided above comprises measuring a level of expression or activity of at least 2, 3, 4, 5, 6, 7, 8, 9, or 10 SMG1-markers described herein.

Further provided herein are methods for achieving one or more clinical endpoints associated with treating or preventing an NMD-dependent or SMG1-dependent tumor described herein. In one embodiment, a patient described herein can show a positive tumor response, such as inhibition of tumor growth or a reduction in tumor size. In certain embodiments, a patient described herein can achieve a Response Evaluation Criteria in Solid Tumors (for example, RECIST 1.1) of complete response, partial response or stable disease after administration of an effective amount of Compound 1 (or a combination therapy described herein). In certain embodiments, a patient described herein can show increased survival without tumor progression. In some embodiments, a patient described herein can show inhibition of disease progression, inhibition of tumor growth, reduction of primary tumor, relief of tumor-related symptoms, inhibition of tumor secreted factors (including tumor secreted hormones, such as those that contribute to carcinoid syndrome), delayed appearance of primary or secondary tumors, slowed development of primary or secondary tumors, decreased occurrence of primary or secondary tumors, slowed or decreased severity of secondary effects of disease, arrested tumor growth and regression of tumors, increased Time To Progression (TTP), increased Progression Free Survival (PFS), and/or increased Overall Survival (OS), among others.

In another embodiment, are methods for increasing the overall survival, objective response rate, time to progression, progression-free survival and/or time-to-treatment failure of a patient having an NMD-dependent or SMG1-dependent tumor described herein comprising administering an effective amount of an NMD-inhibitor or SMG1-inhibitor (e.g., Compound 1) as described herein. In one embodiment, is a method for increasing the overall survival of a patient having an NMD-dependent or SMG1-dependent tumor described herein comprising administering an effective amount of an NMD-inhibitor or SMG1-inhibitor (e.g., Compound 1) as described herein. In one embodiment, is a method for increasing the objective response rate of a patient having an NMD-dependent or SMG1-dependent tumor described herein comprising administering an effective amount of an NMD-inhibitor or SMG1-inhibitor (e.g., Compound 1) as described herein. In one embodiment, is a method for increasing the time to progression of a patient having an NMD-dependent or SMG1-dependent tumor described herein comprising administering an effective amount of an NMD-inhibitor or SMG1-inhibitor (e.g., Compound 1) as described herein. In one embodiment, is a method for increasing the progression-free survival of a patient having an NMD-dependent or SMG1-dependent tumor described herein comprising administering an effective amount of an NMD-inhibitor or SMG1-inhibitor (e.g., Compound 1) as described herein. In one embodiment, is a method for increasing the time-to-treatment failure of a patient having an NMD-dependent or SMG1-dependent tumor described herein comprising administering an effective amount of an NMD-inhibitor or SMG1-inhibitor (e.g., Compound 1) as described herein. In all such embodiments, the NMD-inhibitor or SMG1-inhibitor can be administered as a combination therapy as described herein.

Further provided herein are methods for decreasing mortality of a patient having an NMD-dependent or SMG1-dependent tumor described herein comprising administering an effective amount of an NMD-inhibitor or SMG1-inhibitor (e.g., Compound 1) as described herein.

In certain embodiments described herein the dose or administration schedule of an SMG1-inhibitor is determined by the presence of, or in some embodiments, an aberrant level of expression or activity of an SMG1-marker described herein. For example, modulation of expression or activity of SRSF3 or SRSF6 is used to determine the effective dose of an SMG1-inhibitor. In one embodiment, modulation of expression or activity of SRSF3 or SRSF6 is used to determine the effective dose of Compound 1. In another embodiment, modulation of expression or activity of SRSF3 or SRSF6 is used to determine the administration schedule of an SMG1-inhibitor. In one embodiment, modulation of expression or activity of SRSF3 or SRSF6 is used to determine the administration schedule of Compound 1.

### 5.3 Combination Therapies

Also provided herein are combination therapies that comprise administration of an SMG1-inhibitor and one or more active agents as described herein. The combination therapies provided herein are useful for treating or preventing an NMD-dependent tumor provided herein. The combination therapies useful in the methods provided herein comprise an SMG1-inhibitor as described herein, and one or more of (1) an immuno-oncology treatment, (2) a proteasome inhibitor, (3) a premature termination codon (PTC) read-through compound, (4) an NMD-inhibitor, or (5) a splice factor inhibitor.

In one embodiment, the combination therapy can comprise administration of an SMG1-inhibitor as described herein and an immune-oncology treatment. The immune-oncology treatment can comprise one or more of a CTLA4 inhibitor, a PD-1 inhibitor, a PD-L1 inhibitor, a PD-L2 inhibitor, a LAG3 inhibitor, an antibody to B7-H3, a TIM-3 inhibitor, an OX40 agonist, a CXCR4 inhibitor, a CSF1R inhibitor, an ICOS inhibitor, a Toll-like receptor agonist, a 41-BB agonist, a GITR agonist, a CD27 agonist, a CD40 agonist, an IDO inhibitor, an IDO1-derived peptide, or an IDO inhibitor.

In one embodiment, the combination therapy comprises an immune-oncology treatment that comprises one or more of a CTLA4 antibody, a PD-1 antibody, a PD-L1 antibody, or an IDO inhibitor including, but not limited to those described below. For example, in one embodiment, the combination therapy described herein comprises Compound 1 and one or more of ipilimumab, tremelimumab, pembrolizumab, nivolumab, pidilizumab, atezolizumab, durvalumab, avelumab, indoximod or epacadostat.

In one embodiment the combination therapy comprises a CTLA4 inhibitor. A “CTLA-4 inhibitor” refers to a moiety (e.g., compound, nucleic acid, polypeptide, antibody) that decreases, inhibits, blocks, abrogates or interferes with the activity or binding of CTLA-4 (e.g., Cytotoxic T-Lymphocyte-Associated Protein 4; CTLA-4; GI: 49904741), including variants, isoforms, species homologs of human CTLA-4 (e.g., mouse). Examples of CTLA-4 inhibitors include, but are not limited to, those described in U.S. Pat. Nos. 5,811,097; 5,811,097; 5,855,887; 6,051,227; 6,207,157; 6,682,736; 6,984,720; and 7,605,238, all of which are incorporated herein in their entireties. In one embodiment, the CTLA-4 inhibitor is tremelimumab (also known as ticilimumab or CP-675,206). In another embodiment, the CTLA-4 inhibitor is ipilimumab (also known as MDX-010 or MDX-101). Ipilimumab is a fully human monoclonal IgG antibody that binds to CTLA-4. Ipilimumab is marketed under the trade name Yervoy™.

In one embodiment, the combination therapy comprises a PD-1 inhibitor. A “PD-1 inhibitor” refers to a moiety (e.g., compound, nucleic acid, polypeptide, antibody) that decreases, inhibits, blocks, abrogates or interferes with the activity or expression of PD-1 (e.g., Programmed Cell Death Protein 1; PD-1 (CD279); GI: 145559515), including variants, isoforms, species homologs of human PD-1 (e.g., mouse) and when an antibody, analogs that have at least one common epitope with PD-1. A PD-1 inhibitor includes molecules and macromolecules such as, for example, compounds, nucleic acids, polypeptides, antibodies, peptibodies, diabodies, minibodies, nanobodies, single-chain variable fragments (ScFv), and functional fragments or variants thereof. A PD-1 inhibitor as used herein can refer to any moiety that antagonizes PD-1 activity or expression. PD-1 inhibitor efficacy is measured, for example, by its inhibitor concentration at 50% (half-maximal inhibitor concentration or IC₅₀).

In one embodiment, the combination therapy comprises a PD-L1 inhibitor. A “PD-L1 inhibitor” refers to a moiety (e.g., compound, nucleic acid, polypeptide, antibody) that decreases, inhibits, blocks, abrogates or interferes with the activity or binding of PD-L1 to its receptor, PD-1, or expression of PD-L1 (e.g., Programmed Cell Death 1 Ligand; PD-L1 (CD274); GI: 30088843), including variants, isoforms, species homologs of human PD-L1 (e.g., mouse) and when an antibody, analogs that have at least one common epitope with PD-L1. A PD-L1 inhibitor includes molecules and macromolecules such as, for example, compounds (small molecule compounds), nucleic acids, polypeptides, antibodies, peptibodies, diabodies, minibodies, single-domain antibodies or nanobodies, single-chain variable fragments (ScFv), and fragments or variants thereof. A PD-L1 inhibitor as used herein can refer to any moiety that antagonizes PD-L1 activity, its binding to PD-1, or its expression. PD-L1 inhibitor efficacy is measured, for example, by its inhibitor concentration at 50% (half-maximal inhibitor concentration or IC₅₀).

Examples of PD-1/PD-L1 inhibitors include, but are not limited to, those described in U.S. Pat. Nos. 7,488,802; 7,943,743; 8,008,449; 8,168,757; 8,217,149, and PCT Patent Application Publication Nos. WO2003042402, WO2008156712, WO2010089411, WO2010036959, WO2011066342, WO2011159877, WO2011082400, and WO2011161699, all of which are incorporated herein in their entireties. Further non-limiting examples include agents known in the art such as “nivolumab,” “pembrolizumab,” “pidilizumab,” “AMP-224,” “REGN2810,” “PDR 001,” “MEDI0680,” “durvalumab,” “avelumab,” “atezolizumab,” “BMS-936559,” “STI-A1010,” “STI-A1011,” “STI-A1012,” “STI-A1013,” “STI-A1014,” and “STI-A1015” which are herein used in accordance with their plain and ordinary meaning as understood in the art.

In one embodiment, the PD-1 inhibitor is selected from the group consisting of pembrolizumab, nivolumab, pidilizumab, REGN2810, BGB-A317, PDR001, AMP-224, CT-011, MDX-1106, AMP-514, and MEDI-0680. In one embodiment, the combination therapy comprises an SMG1-inhibitor described herein (e.g., Compound 1) and pembrolizumab, nivolumab, or pidilizumab.

In one embodiment, the PD-L1 inhibitor is selected from the group consisting of atezolizumab (MPDL3280A), durvalumab (MEDI4736), avelumab (MSB0010718C), BMS-936559 (MDX-1105), PDL1 HSC, and TOCA-531. In one embodiment, the combination therapy can comprise an SMG1-inhibitor described herein (e.g., Compound 1) and atezolizumab, durvalumab, or avelumab. In one embodiment, the combination therapy comprises an SMG1-inhibitor described herein (e.g., Compound 1) and durvalumab.

In one embodiment, the combination therapy comprises a PD-L2 inhibitor. A “PD-L2 inhibitor” refers to a moiety (e.g., compound, nucleic acid, polypeptide, antibody) that decreases, inhibits, blocks, abrogates or interferes with the activity or expression of PD-2 (Programmed Cell Death 1 Ligand 2; PD-L2 (CD273); GI:109731119), including variants, isoforms, species homologs of human PD-L2 (e.g., mouse). In one embodiment, the PD-L2 inhibitor is rHIgM12B7A or AMP-224/B7-DC Fc. In one embodiment, the PD-L2 inhibitor is AMP-224.

In one embodiment, the combination therapy comprises a LAG3 inhibitor. A “LAG3 inhibitor” refers to a moiety (e.g., compound, nucleic acid, polypeptide, antibody) that decreases, inhibits, blocks, abrogates or interferes with the activity or expression of LAG3 (Lymphocyte Activation Gene-3; LAG-3; GI:15617341), including variants, isoforms, species homologs of human LAG-3 (e.g., mouse). In one embodiment, the LAG-3 inhibitor is IMP321, BMS-986016, or MGA271. In one embodiment, the LAG3 inhibitor is selected from the group consisting of IMP321 and BMS-986016.

In one embodiment, the combination therapy comprises an antibody to B7-H3 where the antibody is MGA271.

In one embodiment, the combination therapy comprises a TIM-3 inhibitor. A “TIM3 inhibitor” refers to a moiety (e.g., compound, nucleic acid, polypeptide, antibody) that decreases, inhibits, blocks, abrogates or interferes with the activity or expression of TIM3 (T-cell immunoglobulin domain and mucin domain 3; TIM-3; GI: 397787781), including variants, isoforms, species homologs of human TIM-3 (e.g., mouse). (Fourcade et al., J. Exp. Med., 2010, 207, 2175-86; Sakuishi et al., J. Exp. Med., 2010, 207, 2187-94). In one embodiment, the TIM-3 inhibitor is an anti-TIM-3 antibody.

In one embodiment, the combination therapy comprises an OX40 agonist. An “OX40 agonist” refers to a moiety (e.g., compound, nucleic acid, polypeptide, antibody) that decreases, inhibits, blocks, abrogates or interferes with the activity or expression of OX40 (Tumor Necrosis Factor Receptor Superfamily Member 4; OX40 (CD134); GI:4507579), including variants, isoforms, species homologs of human OX40 (e.g., mouse). In one embodiment, the OX40 agonist is MEDI6469.

In one embodiment, the combination therapy comprises a CXCR4 inhibitor. A “CXCR4 inhibitor” refers to a moiety (e.g., compound, nucleic acid, polypeptide, antibody) that decreases, inhibits, blocks, abrogates or interferes with the activity or expression of CXCR4 (C-X-C Chemokine Receptor Type 4; CXCR4 (CD184); GI: 3059120), including variants, isoforms, species homologs of human CXCR4 (e.g., mouse). Exemplary CXCR4 inhibitors include ulocuplumab, RPI-MN, LY-2510924, Burixafor, BKT-140, Balizafortide, X4P-001, F-50067, X4P-002, PF-06747143, and GMI-1359. In one embodiment, the CXCR4 inhibitor is selected from the group consisting of ulocuplumab, RPI-MN, LY-2510924, Burixafor, BKT-140, Balizafortide, X4P-001, F-50067, X4P-002, PF-06747143, and GMI-1359.

In one embodiment, the combination therapy comprises a CSF1R inhibitor. A “CSF1R inhibitor” refers to a moiety (e.g., compound, nucleic acid, polypeptide, antibody) that decreases, inhibits, blocks, abrogates or interferes with the activity or expression of CSF1R (Macrophage Colony-Stimulating Factor 1 Receptor; CSF1R (CD115); GI:569026720), including variants, isoforms, species homologs of human CSF1R (e.g., mouse). Exemplary CSF1R inhibitors include pacritinib, FPA-008, ENMD-2076, Emactuzumab, IMC-CS4, AMG-820, TG-3003, DCC-3014, and AC-708. In one embodiment, the CSF1R inhibitor is selected from the group consisting of pacritinib, FPA-008, ENMD-2076, Emactuzumab, IMC-CS4, AMG-820, TG-3003, DCC-3014, and AC-708.

In one embodiment, the combination therapy comprises an ICOS inhibitor. An “ICOS inhibitor” refers to a moiety (e.g., compound, nucleic acid, polypeptide, antibody) that decreases, inhibits, blocks, abrogates or interferes with the activity or expression of ICOS (Inducible T-cell Costimulator Precursor; ICOS (CD278); GI:15029518), including variants, isoforms, species homologs of human ICOS (e.g., mouse). In one embodiment, the ICOS inhibitor is AMG-557.

In one embodiment, the combination therapy comprises a Toll-like receptor agonist. A “Toll-like receptor agonist” or “TLR agonist” refers to a moiety (e.g., compound, nucleic acid, polypeptide, antibody) that decreases, inhibits, blocks, abrogates or interferes with the activity or expression of a Toll-like receptor (e.g., TLR1, TLR2, TLR3, TLR4, TLR5, TLR6, TLR7, TLR8, TLR9, or TLR10), including variants, isoforms, species homologs of human TLR (e.g., mouse). In one embodiment, the TLR is TLR4. In one embodiment, the TLR is TLR7. In one embodiment, the TLR is TLR8. In one embodiment, the TLR is TLR9. Exemplary Toll-like receptor agonists include MGN-1703, SD-101, Poly-ICLC, Motolimod, MGN-1703, IMO-2055, Imiquimod, ID-G100, ID-CMB305, GNKG-168, G305, TMX-202, EnanDim, VTX-2337, RGIC.1, RGIC.L1, MEDI-9197, IMO-2125, BB-001, BB-006, BB-007, BB-009, OPN-305, IMO-8400. In one embodiment, the Toll-like receptor agonist is selected from the group consisting of MGN-1703, SD-101, Poly-ICLC, Motolimod, MGN-1703, IMO-2055, Imiquimod, ID-G100, ID-CMB305, GNKG-168, G305, TMX-202, EnanDim, VTX-2337, RGIC.1, RGIC.L1, MEDI-9197, IMO-2125, BB-001, BB-006, BB-007, BB-009, OPN-305, and IMO-8400.

In one embodiment, the combination therapy comprises a 41-BB agonist. A “41-BB” agonist refers to a moiety (e.g., compound, nucleic acid, polypeptide, antibody) that decreases, inhibits, blocks, abrogates or interferes with the activity or expression of 41-BB (Tumor Necrosis Factor Receptor Superfamily Member 9; 41-BB (CD137); GI:5730095), including variants, isoforms, species homologs of human 41-BB (e.g., mouse). Exemplary 41-BB agonists include ulocuplumab, RPI-MN, LY-2510924, Burixafor, BKT-140, Balizafortide, X4P-001, F-50067, X4P-002, PF-06747143, and GMI-1359. In one embodiment, the 41-BB agonist is selected from the group consisting of PF-05082566 and Urelumab (BMS-663513). In one embodiment, the combination therapy comprises an SMG1-inhibitor described herein (e.g., Compound 1) and urelumab.

In one embodiment, the combination therapy comprises a GITR agonist. A “GITR agonist” refers to a moiety (e.g., compound, nucleic acid, polypeptide, antibody) that decreases, inhibits, blocks, abrogates or interferes with the activity or expression of GITR (Tumor Necrosis Factor Receptor Superfamily Member 18 Isoform 1; TNFRSF18, GITR (CD357); GI:4759246), including variants, isoforms, species homologs of human GITR (e.g., mouse). In one embodiment, the GITR agonist is TRX-518 or MK-4166. In one embodiment, the GITR agonist is selected from the group consisting of TRX-518 and MK-4166.

In one embodiment, the combination therapy comprises a CD27 agonist. A “CD27 agonist” refers to a moiety (e.g., compound, nucleic acid, polypeptide, antibody) that decreases, inhibits, blocks, abrogates or interferes with the activity or expression of CD27 (CD27 Antigen; CD27; GI 4507587), including variants, isoforms, species homologs of human CD27 (e.g., mouse). In one embodiment, the CD27 agonist comprises CDX-1127.

In one embodiment, the combination therapy comprises a CD40 agonist. A “CD40 agonist” refers to a moiety (e.g., compound, nucleic acid, polypeptide, antibody) that decreases, inhibits, blocks, abrogates or interferes with the activity or expression of CD40 (Tumor Necrosis Factor Receptor Superfamily Member 5 Isoform 1; TNFRSF5 (CD40); GI:4507581), including variants, isoforms, species homologs of human CD40 (e.g., mouse). In one embodiment, the anti-CD40 antibody is CP-870,893.

In one embodiment, the combination therapy comprises an IDO inhibitor. An “IDO inhibitor” refers to a moiety (e.g., compound, nucleic acid, polypeptide, antibody) that decreases, inhibits, blocks, abrogates or interferes with the activity or expression of IDO (indoleamine 2,3-dioxygenase 1; IDO; GI:4504577), including variants, isoforms, species homologs of human IDO (e.g., mouse). Exemplary IDO inhibitors include GDC-0919, 1-methyltryptophan (indoximod), epacadostat (INCB024360), NLG919 (RG6078), IDO1-derived peptide, CRD1152, IDO inhibitors ((GBR-830, OX86, Fc-OX4OL, MOXR0916, RG7888, GSK3174998, and MEDI6383. In one embodiment, the IDO inhibitor is selected from the group consisting of GDC-0919, 1-methyltryptophan (indoximod), epacadostat (INCB024360), and NLG919 (RG6078). In one embodiment, the combination therapy can comprise an IDO inhibitor where the IDO inhibitor is selected from the group consisting of GBR-830, OX86, Fc-OX40L, MOXR0916, RG7888, GSK3174998, and MEDI6383. In one embodiment, the combination therapy comprises an SMG1-inhibitor described herein (e.g., Compound 1) and indoximod or epacadostat. In one embodiment, the combination therapy comprises an IDO1-derived peptide where the IDO1-derived peptide is CRD1152.

In another embodiment, the combination therapy comprises an SMG1-inhibitor or a tautomer, solvate, hydrate, co-crystal, clathrate, polymorph, or pharmaceutically acceptable salt thereof as described herein and a proteasome inhibitor. The proteasome inhibitor is selected from the group consisting of bortezomib, carfilzomib, Oprozomib, Ixazomib, Delanzomib, and Marizomib. Such combinations can be useful in treating, for example multiple myeloma. One exemplary combination therapy comprises Compound 1, or a tautomer, solvate, hydrate, co-crystal, clathrate, polymorph, or pharmaceutically acceptable salt thereof, and Marizomib. Another exemplary combination therapy comprises Compound 1, or a tautomer, solvate, hydrate, co-crystal, clathrate, polymorph, or pharmaceutically acceptable salt thereof, and bortezomib (or carflizomib). Still another exemplary combination therapy comprises Compound 1, or a tautomer, solvate, hydrate, co-crystal, clathrate, polymorph, or pharmaceutically acceptable salt thereof, and ixazomib.

In another embodiment, the combination therapy comprises an SMG1-inhibitor or a tautomer, solvate, hydrate, co-crystal, clathrate, polymorph, or pharmaceutically acceptable salt thereof as described herein and a BCL2 inhibitor. Such combinations can be useful in treating, for example CLL. One exemplary combination therapy comprises Compound 1, or a tautomer, solvate, hydrate, co-crystal, clathrate, polymorph, or pharmaceutically acceptable salt thereof, and venetoclax.

In another embodiment, the combination therapy comprises an SMG1-inhibitor or a tautomer, solvate, hydrate, co-crystal, clathrate, polymorph, or pharmaceutically acceptable salt thereof as described herein and an AR antagonist. Exemplary AR antagonists include, but are not limited to, flutamide, nilutamide, bicalutamide, enzalutamide, apalutamide, cyproterone acetate, megestrol acetate, chlormadinone acetate, spironolactone, canrenone, drospirenone, ketoconazole, topilutamide (fluridil), and cimetidine. Such combinations can be useful in treating, for example prostate cancer. One exemplary combination therapy comprises Compound 1, or a tautomer, solvate, hydrate, co-crystal, clathrate, polymorph, or pharmaceutically acceptable salt thereof, and enzalutamide.

In still another embodiment, the combination therapy comprises an SMG1-inhibitor or a tautomer, solvate, hydrate, co-crystal, clathrate, polymorph, or pharmaceutically acceptable salt thereof as described herein and temozolomide for treating glioblastoma (e.g. GBM). The combination can further include radiation therapy.

In still another embodiment, the combination therapy comprises an SMG1-inhibitor or a tautomer, solvate, hydrate, co-crystal, clathrate, polymorph, or pharmaceutically acceptable salt thereof as described herein and a compound having the formula 3-(5-amino-2-methyl-4-oxoquinazolin-3(4H)-yl)piperidine-2,6-dione, for treating DLBCL. In certain embodiments, the compound having the formula 3-(5-amino-2-methyl-4-oxoquinazolin-3(4H)-yl)piperidine-2,6-dione is a hydrochloride salt of 3-(5-amino-2-methyl-4-oxo-4H-quinazolin-3-yl)-piperidine-2,6-dione, or an enantiomer or a mixture of enantiomers thereof or a pharmaceutically acceptable solvate, hydrate, co-crystal, clathrate, or polymorph thereof. The compound having the formula 3-(5-amino-2-methyl-4-oxoquinazolin-3(4H)-yl)piperidine-2,6-dione can be prepared according to the methods described in U.S. Pat. No. 7,635,700.

In yet another embodiment, the combination therapy comprises an SMG1-inhibitor or a tautomer, solvate, hydrate, co-crystal, clathrate, polymorph, or pharmaceutically acceptable salt thereof as described herein and a PTC read-through compound. In one embodiment, the PCT read-through compound is G418.

In still another embodiment, the combination therapy comprises an SMG1-inhibitor or a tautomer, solvate, hydrate, co-crystal, clathrate, polymorph, or pharmaceutically acceptable salt thereof as described herein and an NMD-inhibitor. In one embodiment, the NMD-inhibitor is selected from the group consisting of NMDi14 and NMDI 1.

In still another embodiment, the combination therapy comprises an SMG1-inhibitor or a tautomer, solvate, hydrate, co-crystal, clathrate, polymorph, or pharmaceutically acceptable salt thereof as described herein and a splice factor inhibitor. In one embodiment, the splice factor inhibitor is selected from the group consisting of H3B8800, DDD00107587 (madrasin), FR901464, meayamycin, meayamycin B, sudemycins, spliceostatin A, herboxidiene, pladienolides from Streptomyces platensis, E7107, GEX1 from Streptomyces sp, isoginkgetin, clotrimazole, flunarizine, chlorhexidine, 1,4-naphthoquinones, 1,4-heterocyclic quinones, tetracarcin A, indole, napththazarin, topoisomerase I inhibitor (NM-506), benzothiazolecardiotonic steroid (digitoxin), protein phosphatase inhibitors (okadaic acid, tautomycin, microcystin-LR, sodium vanadate and pseudocanthardins), amiloride, and N-Palmitoyl-L-leucine. In certain embodiments, the combination therapies described herein comprise Compound 1.

### 5.4 Pharmaceutical Compositions and Routes of Administration

Provided herein are compositions and combination therapies comprising an effective amount of an NMD-inhibitor or SMG1-inhibitor, and compositions and combination therapies such as those described herein comprising an effective amount of an SMG1-inhibitor and a pharmaceutically acceptable carrier or vehicle. In some embodiments, the pharmaceutical compositions described herein are suitable for oral, parenteral, mucosal, transdermal or topical administration. In one embodiment, the SMG1-inhibitor is Compound 1.

The SMG1-inhibitors can be administered to a patient orally or parenterally in the conventional form of preparations, such as capsules, microcapsules, tablets, granules, powder, troches, pills, suppositories, injections, suspensions and syrups. Suitable formulations can be prepared by methods commonly employed using conventional, organic or inorganic additives, such as an excipient (e.g., sucrose, starch, mannitol, sorbitol, lactose, glucose, cellulose, talc, calcium phosphate or calcium carbonate), a binder (e.g., cellulose, methylcellulose, hydroxymethylcellulose, polypropylpyrrolidone, polyvinylpyrrolidone, gelatin, gum arabic, polyethyleneglycol, sucrose or starch), a disintegrator (e.g., starch, carboxymethylcellulose, hydroxypropylstarch, low substituted hydroxypropylcellulose, sodium bicarbonate, calcium phosphate or calcium citrate), a lubricant (e.g., magnesium stearate, light anhydrous silicic acid, talc or sodium lauryl sulfate), a flavoring agent (e.g., citric acid, menthol, glycine or orange powder), a preservative (e.g., sodium benzoate, sodium bisulfate, methylparaben or propylparaben), a stabilizer (e.g., citric acid, sodium citrate or acetic acid), a suspending agent (e.g., methylcellulose, polyvinyl pyrroliclone or aluminum stearate), a dispersing agent (e.g., hydroxypropylmethylcellulose), a diluent (e.g., water), and base wax (e.g., cocoa butter, white petrolatum or polyethylene glycol). The effective amount of the SMG1-inhibitor in the pharmaceutical composition can be at a level that will exercise the desired effect; for example, about 0.005 mg/kg of a patient's body weight to about 10 mg/kg of a patient's body weight in unit dosage for both oral and parenteral administration.

In general, the SMG1-inhibitors can be administered one to four times a day in a dose of about 0.005 mg/kg of a patient's body weight to about 10 mg/kg of a patient's body weight in a patient, but the above dosage may be properly varied depending on the age, body weight and medical condition of the patient and the type of administration. In one embodiment, the dose is about 0.01 mg/kg of a patient's body weight to about 5 mg/kg of a patient's body weight, about 0.05 mg/kg of a patient's body weight to about 1 mg/kg of a patient's body weight, about 0.1 mg/kg of a patient's body weight to about 0.75 mg/kg of a patient's body weight, about 0.25 mg/kg of a patient's body weight to about 0.5 mg/kg of a patient's body weight, or about 0.007 mg/kg of a patient's body weight to about 1.7 mg/kg of patient's body weight. In one embodiment, one dose is given per day. In another embodiment, two doses are given per day. In any given case, the amount of the SMG1-inhibitor described herein administered will depend on such factors as the solubility of the active component, the formulation used and the route of administration.

In another embodiment, provided herein are methods for the treatment or prevention of an NMD-dependent tumor or an SMG1-dependent cancer, comprising the administration of about 0.375 mg/day to about 750 mg/day, about 0.75 mg/day to about 375 mg/day, about 3.75 mg/day to about 75 mg/day, about 7.5 mg/day to about 55 mg/day, about 18 mg/day to about 37 mg/day, about 0.5 mg/day to about 60 mg/day, or about 0.5 mg/day to about 128 mg/day of an SMG1-inhibitor described herein to a patient having an NMD-dependent tumor or an SMG1-dependent cancer. In another embodiment, provided herein are methods for the treatment or prevention of an NMD-dependent tumor or an SMG1-dependent cancer, comprising the administration of about 0.5 mg/day to about 1200 mg/day, about 10 mg/day to about 1200 mg/day, about 100 mg/day to about 1200 mg/day, about 400 mg/day to about 1200 mg/day, about 600 mg/day to about 1200 mg/day, about 400 mg/day to about 800 mg/day or about 600 mg/day to about 800 mg/day of an SMG1-inhibitor described herein to a patient having an NMD-dependent tumor or an SMG1-dependent cancer. In a particular embodiment, the methods disclosed herein comprise the administration of 0.5 mg/day, 1 mg/day, 2 mg/day, 4 mg/day, 8 mg/day, 16 mg/day, 20 mg/day, 25 mg/day, 30 mg/day, 45 mg/day, 60 mg/day, 90 mg/day, 120 mg/day or 128 mg/day of an SMG1-inhibitor described herein to a patient having an NMD-dependent tumor or an SMG1-dependent cancer.

In another embodiment, provided herein are unit dosage formulations that comprise between about 0.1 mg and about 2000 mg, about 1 mg and 200 mg, about 35 mg and about 1400 mg, about 125 mg and about 1000 mg, about 250 mg and about 1000 mg, or about 500 mg and about 1000 mg of an SMG1-inhibitor described herein.

In a particular embodiment, provided herein are unit dosage formulation comprising about 0.1 mg, 0.25 mg, 0.5 mg, 1 mg, 5 mg, 7.5 mg, 10 mg, 15 mg, 20 mg, 30 mg, 45 mg, 50 mg, 60 mg, 75 mg, 100 mg, 125 mg, 150 mg, 200 mg, 250 mg, 300 mg, 400 mg, 600 mg or 800 mg of an SMG1-inhibitor described herein.

In another embodiment, provided herein are unit dosage formulations that comprise 0.1 mg, 0.25 mg, 0.5 mg, 1 mg, 2.5 mg, 5 mg, 7.5 mg, 10 mg, 15 mg, 20 mg, 30 mg, 35 mg, 50 mg, 70 mg, 100 mg, 125 mg, 140 mg, 175 mg, 200 mg, 250 mg, 280 mg, 350 mg, 500 mg, 560 mg, 700 mg, 750 mg, 1000 mg or 1400 mg of an SMG1-inhibitor described herein. In a particular embodiment, provided herein are unit dosage formulations that comprise 5 mg, 7.5 mg, 10 mg, 15 mg, 20 mg, 30 mg, 45 mg or 60 mg of an SMG1-inhibitor described herein.

In one embodiment, SMG1-inhibitor described herein is administered once, twice, three, four or more times daily.

In one embodiment, SMG1-inhibitor described herein is administered orally for reasons of convenience. In one embodiment, when administered orally, an SMG1-inhibitor is administered with a meal and water. In another embodiment, the SMG1-inhibitor described herein is dispersed in water or juice (e.g., apple juice or orange juice) and administered orally as a suspension. In another embodiment, when administered orally, an SMG1-inhibitor described herein is administered in a fasted state.

The SMG1-inhibitor described herein can also be administered intradermally, intramuscularly, intraperitoneally, percutaneously, intravenously, subcutaneously, intranasally, epidurally, sublingually, intracerebrally, intravaginally, transdermally, rectally, mucosally, by inhalation, or topically to the ears, nose, eyes, or skin. The mode of administration can be left to the discretion of the health-care practitioner, and can depend in-part upon the site of the medical condition.

In one embodiment, provided herein are capsules containing an SMG1-inhibitor described herein without an additional carrier, excipient or vehicle.

In another embodiment, provided herein are compositions, comprising an effective amount of an SMG1-inhibitor described herein and a pharmaceutically acceptable carrier or vehicle, wherein a pharmaceutically acceptable carrier or vehicle comprises an excipient, diluent, or a mixture thereof. In one embodiment, the composition is a pharmaceutical composition.

The compositions can be in the form of tablets, chewable tablets, capsules, solutions, parenteral solutions, troches, suppositories and suspensions and the like. Compositions can be formulated to contain a daily dose, or a convenient fraction of a daily dose, in a dosage unit, which may be a single tablet or capsule or convenient volume of a liquid. In one embodiment, the solutions are prepared from water-soluble salts, such as the hydrochloride salt. In general, all of the compositions are prepared according to known methods in pharmaceutical chemistry. Capsules can be prepared by mixing an SMG1-inhibitor described herein with a suitable carrier or diluent and filling the proper amount of the mixture in capsules. The usual carriers and diluents comprise, but are not limited to, inert powdered substances such as starch of many different kinds, powdered cellulose, especially crystalline and microcrystalline cellulose, sugars such as fructose, mannitol and sucrose, grain flours and similar edible powders.

Tablets can be prepared by direct compression, by wet granulation, or by dry granulation. Their formulations usually incorporate diluents, binders, lubricants and disintegrators as well as the compound. Typical diluents comprise, for example, various types of starch, lactose, mannitol, kaolin, calcium phosphate or sulfate, inorganic salts such as sodium chloride and powdered sugar. Powdered cellulose derivatives are also useful. In one embodiment, the pharmaceutical composition is lactose-free. Typical tablet binders are substances such as starch, gelatin and sugars such as lactose, fructose, glucose and the like. Natural and synthetic gums are also convenient, including acacia, alginates, methylcellulose, polyvinylpyrrolidine and the like. Polyethylene glycol, ethylcellulose and waxes can also serve as binders.

A lubricant might be necessary in a tablet formulation to prevent the tablet and punches from sticking in the die. The lubricant can be chosen from such slippery solids as talc, magnesium and calcium stearate, stearic acid and hydrogenated vegetable oils. Tablet disintegrators are substances that swell when wetted to break up the tablet and release the compound. They comprise starches, clays, celluloses, algins and gums. More particularly, corn and potato starches, methylcellulose, agar, bentonite, wood cellulose, powdered natural sponge, cation-exchange resins, alginic acid, guar gum, citrus pulp and carboxymethyl cellulose, for example, is used as well as sodium lauryl sulfate. Tablets can be coated with sugar as a flavor and sealant, or with film-forming protecting agents to modify the dissolution properties of the tablet. The compositions can also be formulated as chewable tablets, for example, by using substances such as mannitol in the formulation.

When it is desired to administer an SMG1-inhibitor described herein as a suppository, typical bases can be used. Cocoa butter is a traditional suppository base, which is modified by addition of waxes to raise its melting point slightly. Water-miscible suppository bases comprising, particularly, polyethylene glycols of various molecular weights are in wide use.

The effect of the SMG1-inhibitor described herein can be delayed or prolonged by proper formulation. For example, a slowly soluble pellet of the SMG1-inhibitor described herein can be prepared and incorporated in a tablet or capsule, or as a slow-release implantable device. The technique also comprises making pellets of several different dissolution rates and filling capsules with a mixture of the pellets. Tablets or capsules can be coated with a film that resists dissolution for a predictable period of time. Even the parenteral preparations can be made long-acting, by dissolving or suspending the SMG1-inhibitor described herein in oily or emulsified vehicles that allow it to disperse slowly in the serum.

Administration of the components of a combination therapy as described herein can occur simultaneously or sequentially by the same or different routes of administration. The suitability of a particular route of administration employed for a particular active agent will depend on the active agent itself (e.g., whether it is administered orally without decomposing prior to entering the blood stream) and the cancer being treated.

The route of administration of an SMG1-inhibitor provided herein is independent of the route of administration of a combination active agent combination active agent (e.g., an immuno-oncology treatment, a proteasome inhibitor, a premature termination codon (PTC) read-through compound, an NMD-inhibitor, or a splice factor inhibitor) as described herein. In one embodiment, an SMG1-inhibitor provided herein is administered orally (PO). In another embodiment, an SMG1-inhibitor provided herein is administered intravenously (IV). An SMG1-inhibitor provided herein can be administered orally or intravenously, and the combination active agent is administered orally, parenterally, intraperitoneally, intravenously, intraarterially, transdermally, sublingually, intramuscularly, rectally, transbuccally, intranasally, liposomally, via inhalation, vaginally, intraoccularly, via local delivery by catheter or stent, subcutaneously, intraadiposally, intraarticularly, intrathecally, or in a slow release dosage form. In one embodiment, the combination active agent is administered orally, parenterally, intraperitoneally, intravenously or liposomally. In one embodiment, the combination active agent is administered in accordance with a package insert provided with the agent. The term package insert refers to instructions customarily comprised in commercial packages of medicaments approved by the FDA or a similar regulatory agency of a country other than the USA, which contains information about, for example, the usage, dosage, administration, contraindications, and/or warnings concerning the use of such medicaments. In one embodiment, an SMG1-inhibitor provided herein and a combination active agent provided herein are administered by the same mode of administration, e.g., orally or by IV. In another embodiment, an SMG1-inhibitor provided herein is administered by one mode of administration, e.g., by PO, whereas a combination active agent provided herein is administered by another mode of administration, e.g., IV

A combination active agent (e.g., an immuno-oncology treatment, a proteasome inhibitor, a premature termination codon (PTC) read-through compound, an NMD-inhibitor, or a splice factor inhibitor) described herein for use in a combination therapy described herein can be administered in amounts from about 0.005 to about 2,000 mg per day, from about 0.005 to about 1,000 mg per day, from about 0.01 to about 500 mg per day, from about 0.01 to about 250 mg per day, from about 0.01 to about 100 mg per day, from about 0.1 to about 100 mg per day, from about 0.5 to about 100 mg per day, from about 1 to about 100 mg per day, from about 0.01 to about 50 mg per day, from about 0.1 to about 50 mg per day, from about 0.5 to about 50 mg per day, from about 1 to about 50 mg per day, from about 0.02 to about 25 mg per day, or from about 0.05 to about 10 mg per day. In one embodiment a combination active agent described herein is administered in an amount from about 500 mg to about 2500 mg, 750 mg to about 2250 mg, 1000 mg to about 2000 mg, or about 1200 mg to about 1800 mg.

A combination active agent described herein can be administered in a therapeutically effective amount of about 100, 200, 300, 400, 500, 600, 700, 800, 900, 1000, 1100, 1200, 1300, 1400, 1500, 1600, 1700, 1800, 1900, 2000, 2100, 2200, 2300, 2400, or about 2500 mg. In certain embodiments the combination active agent is administered in a therapeutically effective amount of about 1000, 1250, 1500, 1750, or 2000 mg.

In one embodiment the combination active agent is a PD-L1 inhibitor. In one embodiment the combination active agent is durvalumab. Durvalumab can be administered in an amount from 1 mg to about 2,000 mg per day, from about 100 mg to about 2,000 mg per day, from about 250 mg to about 2,000 mg per day, from about 500 mg to about 2,000 mg per day, 1 mg to about 1,500 mg per day, from about 100 mg to about 1,500 mg per day, from about 250 mg to about 1,500 mg per day, from about 500 mg to about 1,500 mg per day, 1 mg to about 1,000 mg per day, from about 100 mg to about 1,000 mg per day, from about 250 mg to about 1,000 mg per day, from about 500 mg to about 1,000 mg per day, from about 250 mg to about 750 mg per day, or from about 400 mg to about 600 mg per day. In another embodiment the combination active agent is durvalumab administered at an amount of about 100, 250, 500, 1,000, 1,500, or 2,000 mg per day. When the combination active agent is durvalumab it can be administered at a concentration of about 50 mg/mL. In one embodiment, a combination therapy as described herein is co-administered to a patient receiving radiation therapy (e.g., local involved field radiation therapy (IFRT)).

In certain embodiments, the patient to be treated with a combination therapy described herein has not been treated with anticancer therapy prior to the administration the combination therapy. In certain embodiments, the patient to be treated with a combination therapy described herein has been treated one or both of the agents of the combination therapy described herein (e.g., an SMG1-inhibitor described herein or a combination active agent). In certain embodiments, the patient to be treated with a combination therapy described herein has not been treated one or both of the agents of the combination therapy described herein (e.g., an SMG1-inhibitor described herein or a combination active agent). In certain embodiments, the patient to be treated with a combination therapy described herein has been treated with anticancer therapy prior to administration of a compound described herein for use in a combination therapy described herein. In certain embodiments, the patient to be treated with a combination therapy described herein has developed drug resistance to, or has a cancer that is refractory to, at least one anticancer therapy.

Combination active agents provided herein can be administered according to the routes and dosage amounts generally known to a person of ordinary skill in the art. In certain embodiments, a combination active agent described herein is administered in accordance with established protocols known in the art (e.g., marketed compositions).

Pharmaceutical compositions of SMG1-inhibitors described herein, combination active agents, and combination therapies described herein can be supplied as single unit dosage forms. Single unit dosage forms provided herein can be suitable for oral, mucosal (e.g., nasal, sublingual, vaginal, buccal, or rectal), parenteral (e.g., subcutaneous, intravenous, bolus injection, intramuscular, or intra-arterial), topical (e.g., eye drops or other ophthalmic preparations), transdermal, or transcutaneous administration to a patient. Examples of dosage forms include, but are not limited to: tablets; caplets; capsules, such as soft elastic gelatin capsules; cachets; troches; lozenges; dispersions; suppositories; powders; liposomes; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; eye drops or other ophthalmic preparations suitable for topical administration; and sterile solids (e.g., crystalline or amorphous solids) that is reconstituted to provide liquid dosage forms suitable for parenteral administration to a patient.

It is understood that a single unit dosage form as provided herein can refer to a unit where each component of the combination therapy administered can be separately formulated. For example, an SMG1-inhibitor described herein can be formulated for PO administration and separately from a combination active agent which can, for example be formulated for PO or parenteral administration (e.g., IV). It is further understood that where the active ingredients of the combination therapy described herein are conducive to co-formulation, SMG1-inhibitors and combination active agents can be co-formulated (e.g. for PO or parenteral administration) as a single combination dosage form.

### 5.5 Kits

In certain embodiments, provided herein are kits comprising an SMG1-inhibitor.

In other embodiments, provided herein are kits comprising an SMG1-inhibitor described herein and means for monitoring patient response to administration of the SMG1-inhibitor. In certain embodiments, the patient has an NMD-dependent tumor. In particular embodiments, the patient response measured is inhibition of disease progression, inhibition of tumor growth, reduction of primary and/or secondary tumor(s), relief of tumor-related symptoms, improvement in quality of life, delayed appearance of primary and/or secondary tumors, slowed development of primary and/or secondary tumors, decreased occurrence of primary and/or secondary tumors, slowed or decreased severity of secondary effects of disease, arrested tumor growth or regression of tumor.

In other embodiments, provided herein are kits comprising an SMG1-inhibitor described herein and means for measuring of the activity of one or more SMG1-markers in a patient. In certain embodiments, provided herein are kits comprising an SMG1-inhibitor and means for measuring the activity of one or more SMG1-markers as assessed by comparison of the activity of one or more SMG1-markers before, during and/or after administration of the SMG1-inhibitor.

In certain embodiments, the kits provided herein further comprise instructions for use, such as for administering an SMG1-inhibitor described herein or a combination active agent and/or monitoring patient response to administration of an SMG1-inhibitor.

In certain embodiments, the kits provided herein further comprise a combination active agent to be used in combination with the SMG1-inhibitor where the agent is one or more of an immuno-oncology treatment, a proteasome inhibitor, a premature termination codon (PTC) read-through compound, an NMD-inhibitor, or a splice factor inhibitor.

### 6. EXAMPLES

### 6.1 Biological Examples

### 6.1.1 Dose-Response Growth Curves for Cell Lines Treated with Compounds 1 (1-ethyl-7-(2-methyl-6-(1H-1,2,4-triazol-3-yl)pyridin-3-yl)-3,4-dihydropyrazino[2,3-b]pyrazin-2(1H)-one) and 2 (7-(6-(2-hydroxypropan-2-yl)pyridin-3-yl)-1-((trans)-4-methoxycyclohexyl)-3,4-dihydropyrazino[2,3-b]pyrazin-2(1H)-one)

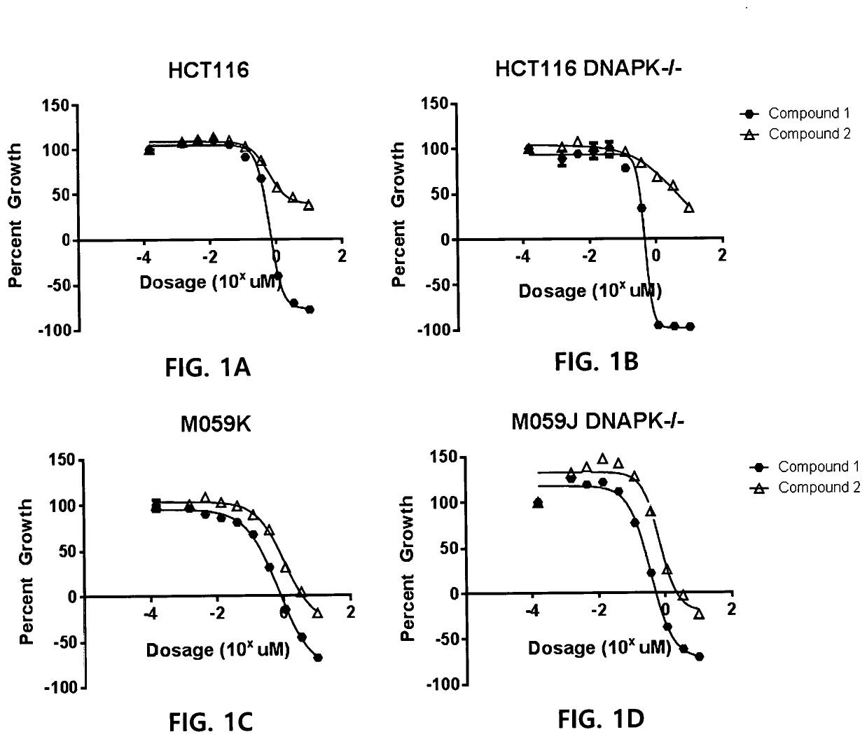

Cell lines: Cell lines were purchased from American Type Culture Collection (ATCC) or the Deutsche Sammlung von Mikroorganismen and Zellkulturen (DSMZ). Cells were cultured according to the vendor's recommendation. Determination of GI50 and Emax or the majority of lines were generated by spotting increasing concentrations of compound (1.5 nM to 10 μM) via an acoustic dispenser (EDCATS-100) into an empty 384-well plate. Compound was spotted in a 10-point serial dilution fashion (3-fold dilution) in duplicate within the plate. The dimethyl sulfoxide (DMSO) concentration was kept constant for a final assay concentration of 0.1% DMSO. For testing, cells were diluted to the appropriate densities and added directly to the compound-spotted 384-well plates. At the time when compound was added (t0), initial cell number was assessed via a viability assay (Cell Titer-Glo) by quantifying the level of luminescence generated by ATP present in viable cells. After 72 hours, cell viability of compound-treated cells was assessed using Cell Titer-Glo (Promega). Cell lines were assayed for growth inhibition by Compound 1 for at least 3 independent tests. Results were then expressed as a GI50, which is the compound concentration required to inhibit cell growth in treated cells to 50% of the growth of the untreated control cells. The GI50 value corrects for the cell count at time zero. For a minority lines, dose response data were generated by using manual dilution series of compound instead of using an acoustic dispenser, and working in a 96 instead of a 384 well format, but otherwise, the protocols were the same. In addition, we calculated the maximum inhibition (Emax) by applying the following test and equation: If T72<T0:100*(T72−T0)/T0. If T72≥T0:100*[(T72−T0)/(V72−T0)] where T72 is the signal for the test compound treated cells at 72h, V72 is the vehicle-treated control measure, and TO is the signal for the compound treated cells at time zero. Results are shown in FIGS. 1A-1D.

Conclusions: The data show that the cellular response to Compound 1 treatment is due to SMG1 inhibition, at least in these cell lines. A dose dependent response to Compound 1 is shown, including cell death (below 0) at higher doses. These effects are not due to inhibition of TORK, since Compound 1 does not have the same effect, nor to inhibition of DNAPK, since the effect occurs in cells without DNAPK.

### 6.1.2 KiNativ Assay

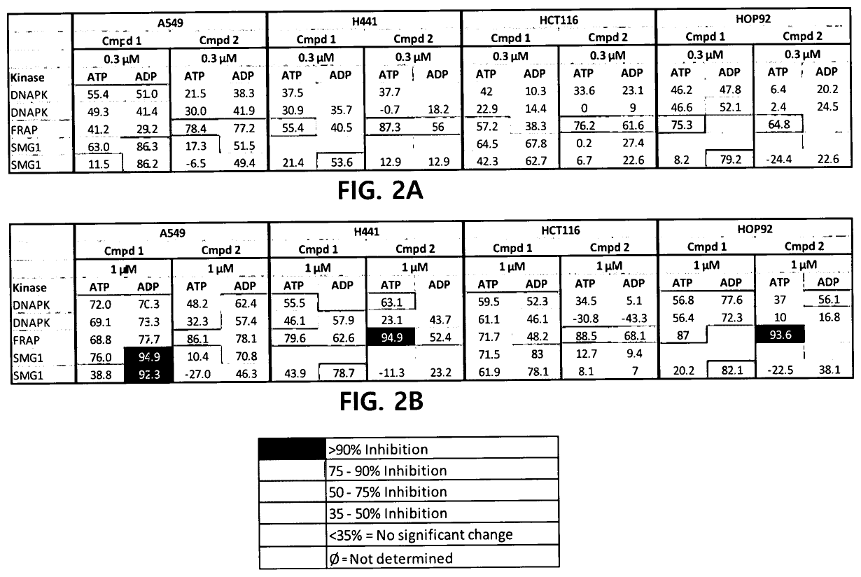

Compound treatment of cells for KiNativ assay: Cell were grown to confluence in appropriate media and treated with compound (Compound 1 (1-ethyl-7-(2-methyl-6-(1H-1,2,4-triazol-3-yl)pyridin-3-yl)-3,4-dihydropyrazino[2,3-b]pyrazin-2(1H)-one) or Compound 2 (7-(6-(2-hydroxypropan-2-yl)pyridin-3-yl)-1-((trans)-4-methoxycyclohexyl)-3,4-dihydropyrazino[2,3-b]pyrazin-2(1H)-one)) at the appropriate concentration in 0.1% DMSO. After 1 h, the medium was aspirated; cells were rinsed once with ice cold PBS, scraped off and transferred to a 15 ml conical tube. Cells were pelleted by centrifugation, the supernatant was discarded and pellets were frozen on dry ice and stored at −80° C. KiNativ assay: Briefly, lysates were prepared from cell pellets and incubated with ADP and ATP probes. After a tryptic digest, the probe labeled peptides were characterized and quantified using targeted Liquid Chromatography—Mass Spectrometry (LC-MS). Comparison of MS signals from treated and untreated cells identified kinases bound by the compounds as shown in FIGS. 2A-2B. Percent inhibition is defined as the reduction of probe binding in the presence of compound, indicating binding of the compound to the kinase.

Conclusion. The data illustrates the target specificity of Compound 1, showing binding to TORK, DNAPK and SMG1, distinguish it from Compound 2, which only binds TORK.

### 6.1.3 Inhibition of SMG1 is Specific to Compound 1 (1-ethyl-7-(2-methyl-6-(1H-1,2,4-triazol-3-yl)pyridin-3-yl)-3,4-dihydropyrazino[2,3-b]pyrazin-2(1H)-one)

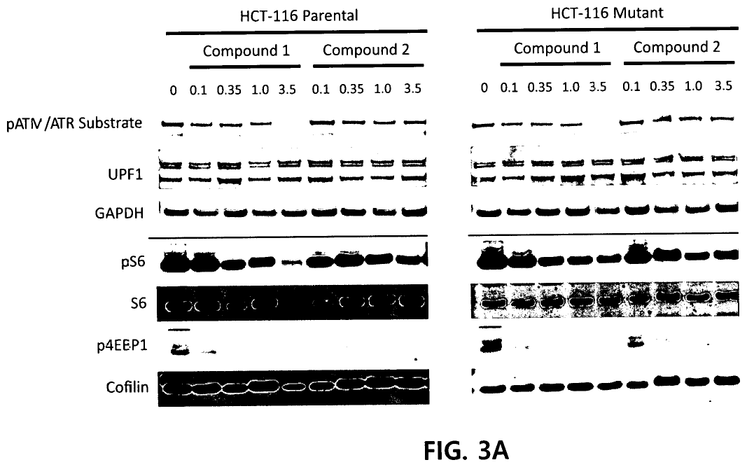

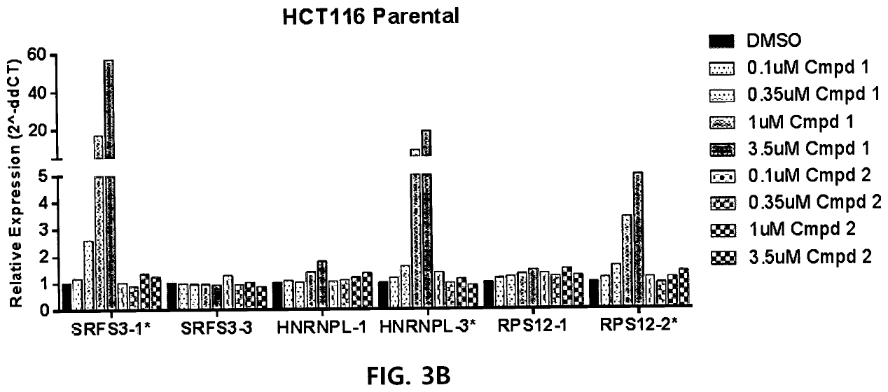

SDS-PAGE and Western blot: Equal amounts of protein were subjected to SDS-PAGE using 3-8% tris-acetate protein gels (Thermo Fisher Scientific # A03755BOX, for DNA-PK, SMG1 and vinculin detection) and 4-12% bis-tris protein gels (Thermo Fisher Scientific # NP0323BOX, for phospho-(Ser/Thr) ATM/ATR substrate, UPF1, GAPDH, S6, phospho-4E-BP1, and cofilin detection). Proteins were electrophoretically transferred to 0.2 μm nitrocellulose membranes (Thermo Fisher Scientific # LC2000) and blocked for 1 hour with Odyssey Blocking Buffer TBS (LI-COR #927-50000). Membranes from 3-8% tris-acetate gels were incubated overnight with antibodies against total DNA-PK (Cell Signaling #12311), phospho DNA-PK (Ser2056) (Abcam # ab124918), total SMG1 (Cell Signaling #9149) and vinculin (Sigma # V9264). Membranes from 4-12% bis-tris gels were incubated overnight with antibodies against phospho-(Ser/Thr) ATM/ATR substrate (Cell Signaling #2851), total UPF1 (Sigma # SAB 1402893), GAPDH (Santa Cruz #47724), total S6 Ribosomal Protein (Santa Cruz #74459), phosphoS6 Ribosomal Protein (Ser240/244) (Cell Signaling #5364), phospho-4E-BP1 (Thr70) (Cell Signaling #9455), and cofilin (Santa Cruz #376476). Membranes were washed with TBST and incubated with IRDye 800CW Goat anti-Rabbit IgG (LI-COR #926-32211) and IRDye 680LT Goat anti-Mouse IgG (LI-COR #926-68020) secondary antibodies for 1 hour. Membranes were washed with TB ST/TBS and scanned on the Odyssey Infrared Imager (LI-COR). Real-Time PCR and NMD assay: Total RNA was prepared using Qiagen's RNeasy Mini kit (Cat #74104) following the manufacturer's instructions. Contaminating gDNA was eliminated by DNase digestion to prevent RT-PCR amplification of any. 2 μg RNA was reverse transcribed into cDNA in a 20 μl reaction using ABI's High Capacity RNA-to-cDNA Kit (Cat #4387406) following the manufacturer's instructions. cDNA was diluted 10× with nuclease-free H₂O and mixed with a final concentration of 200 nM forward and reverse primers, 100 nM taqman probe, lx Taqman PCR master mix according to applied biosystem protocols. Primers for NMD transcripts were designed around exons that were normally subjected to NMD, using 2 primer pairs that would detect either the NMD exon, or as a control the two bordering exons. Inhibition of UPF1 phosphorylation and NMD by Compound 1 was confirmed by Western blot and qPCR analysis as provided in FIGS. 3A-3B.

Conclusion. The data illustrates that in both parental and DNAPK−/− HCT116 cells Compound 1 inhibits TORK (pS6 and p4EBP1) and SMG1 (pUPF1, as measured by antibody to pATM/ATR substrate), while Compound 2 binds to TORK (pS6 and p4EBP1) only. Additionally, Compound 1, but not Compound 2, inhibits SMG1, as measured by upregulation of NMD transcripts, and not the normal version of that transcript.

Normal and NMD RT-PCR Assay Sequences:

| Gene | Primer name | sequence |
|---|---|---|
| SRSF3 | SRSF3_1_F | CGTCGCCCTCGAGATGAT (SEQ ID NO: 1) |
| | SRSF3_1_R | TGACTGGCTAATGCAGATTCAGA (SEQ ID NO: 2) |
| | SRSF3_3_F | CGTCGCCCTCGAGATGAT (SEQ ID NO: 3) |
| | SRSF3_3_R | CGGCTGCGAGAGAAGCTT (SEQ ID NO: 4) |
| SRSF6 | SRSF6_1_F | CGATCGCGACGGCTACA (SEQ ID NO: 5) |
| | SRSF6_1_R | AAAGGCAGTCATCTTAGCCTCAGT (SEQ ID NO: 6) |
| | SRSF6_3_F | ATCGCGACGGCTACAGCTA (SEQ ID NO: 7) |
| | SRSF6_3_R | CGTATTTGTCTCTGCCAGATGTTC (SEQ ID NO: 8) |
| HNRNPL | HNRNPL_For1 | CTTGGGACTACACAAACCCCA (SEQ ID NO: 9) |
| | HNRNPL_Rev1 | GTGGGGCCCTCCATATTCTG (SEQ ID NO: 10) |
| | HNRNPL_For2 | CTGATTGACGGTGTGGTGGA (SEQ ID NO: 11) |
| | HNRNPL_Rev2 | CTCCACCAGTGCTTGTCTCT (SEQ ID NO: 12) |
| | HNRNPL_For3 | CTTGGGACTACACAAACCCCA (SEQ ID NO: 13) |
| | HNRNPL_Rev3 | TGGACGCTTCAACAGTGAGT (SEQ ID NO: 14) |
| RPS12 | RBS12_For1 | CTCATCCACGATGGCCTAGC (SEQ ID NO: 15) |
| | RBS12_Rev1 | CACAGTTGGATGCAAGCACA (SEQ ID NO: 16) |
| | RBS12_For2 | AGGCATGGAGTTCATGGTGTT (SEQ ID NO: 17) |
| | RBS12_Rev2 | CACAGTTGGATGCAAGCACA (SEQ ID NO:18) |
| HNRNPK | HNRNPK_For1 | AGAATGCTGGGGCAGTGATT (SEQ ID NO: 19 |
| | HNRNPK_Rev1 | CACTGCTGTCTGGGACTGAA (SEQ ID NO: 20) |
| | HNRNPK_For2 | AGAATGCTGGGGCAGTGATT (SEQ ID NO: 21) |
| | HNRNPK_Rev2 | TCCAAGGTAGGGATGATTTTCTTCA (SEQ ID NO: 22) |
| NEU1 | NEU1_For1 | GGTCAGCCCAAGCAGGAAAA (SEQ ID NO: 23) |
| | NEU1_Rev1 | GGTTTCGGGCATTGATGACG (SEQ ID NO: 24) |
| | NEU1_For2 | GGTCAGCCCAAGCAGGAAAA (SEQ ID NO: 25) |
| | NEU1-Rev2 | CAGGGTCGAAGGTCACATCA (SEQ ID NO: 26) |
| SF1 | SF1_For1 | GCCTACAGTTATTCCCCCTGG (SEQ ID NO: 27) |
| | SF1_Rev1 | TGTGCGCAGTTTACGAGTCA (SEQ ID NO: 28) |
| | SF1_For2 | GCCTACAGTTATTCCCCCTGG (SEQ ID NO: 29) |
| | SF1_Rev2 | AACTCTCGGGTGTTAAGCCG (SEQ ID NO: 30) |

### 6.1.4 Markers of Inhibition

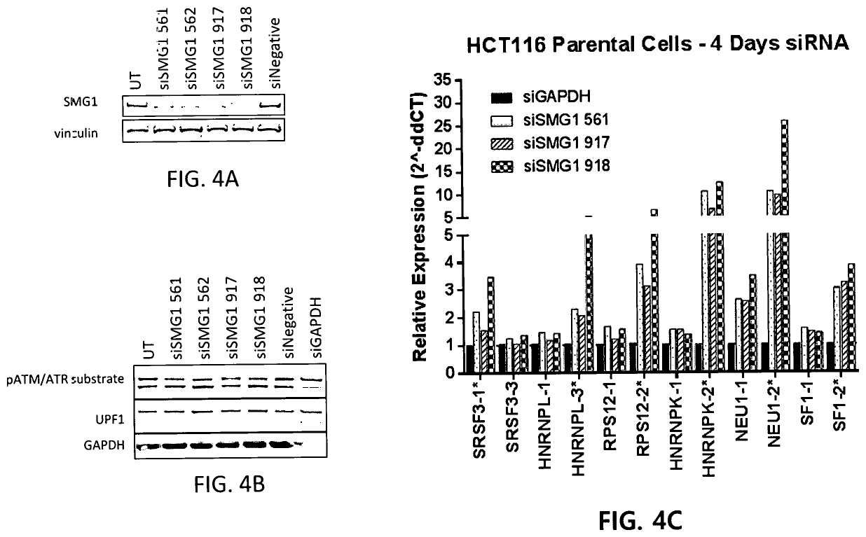

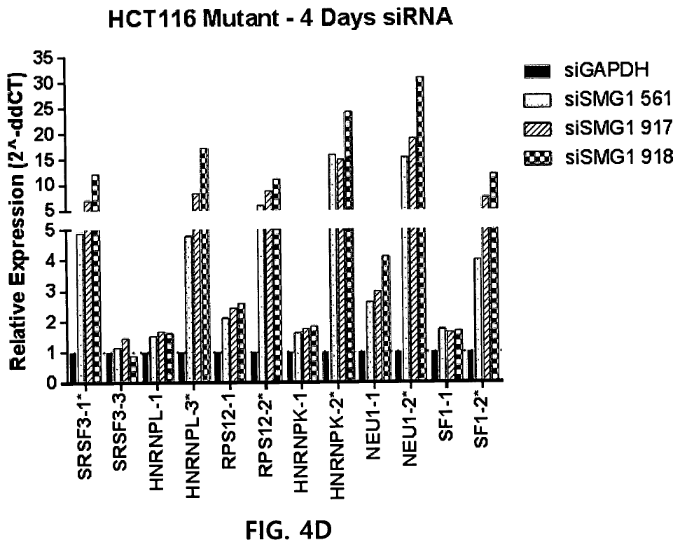

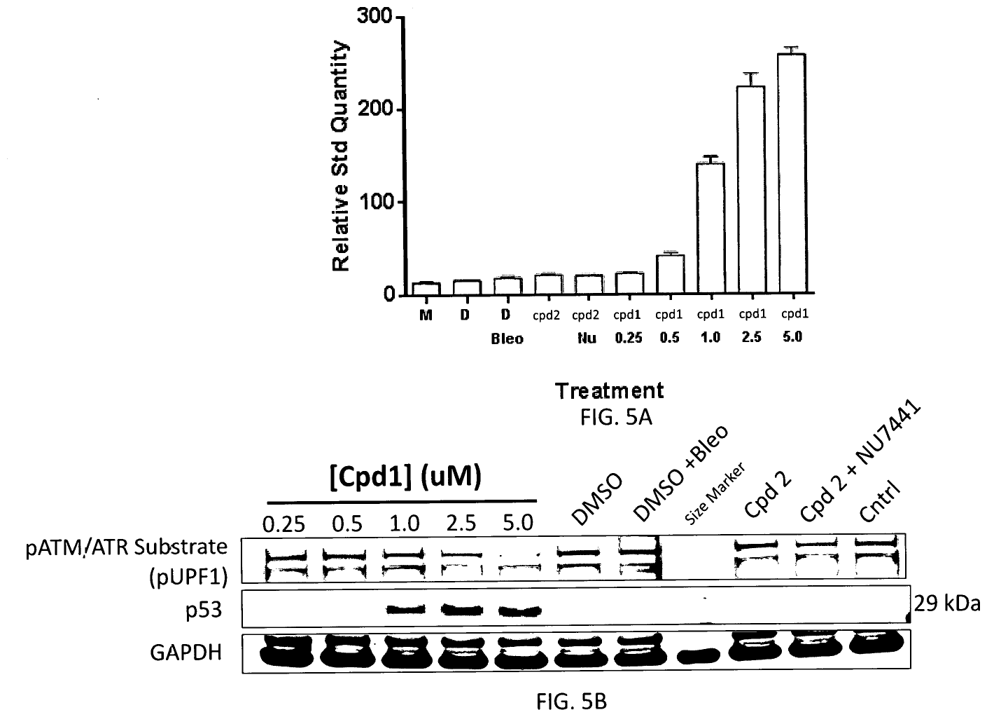

SiRNA assays: siRNAs were ordered from Thermo Fisher Scientific. Four Silencer Select Pre-designed siRNA against SMG1, s223561 (#4392420_3622641652), s223562 (#4392420_3622641696), s22917 (4392420_3622641700) and s22918 (4392420_3622641633) together with a GAPDH positive control siRNA and a negative control siRNA were used. Lipid-mediated transfection was done according to recommendations by the manufacturer, using lipofectamine and 10 nM siRNA. Knock-down was confirmed by Western blot and qPCR analysis as provided in FIGS. 4A-4D.

Conclusion. The data shows that the SMG1 markers used for Compound 1 are appropriate SMG1 markers, since genetic knock-out of SMG1 changes their levels. This is shown by SMG1 knockdown via siRNA, inhibition of the SMG1 marker pUPF1 by SMG1 knockdown (via siRNA) and by the upregulation of NMD transcripts by SMG1 knockdown (via siRNA).

### 6.1.5 Inhibition of SMG1 by Compound 1 Increases Expression of Mutant p53 mRNA and Protein

Calu6 cells were treated with Compound 1, Compound 2, or Compound 2+NU7441 (a reported DNA-PKi) or bleomycin (to induce DNA damage). Expression of the p53 mutant was evaluated at the RNA level (by RT-PCR) or protein level (by western blot). pUPF1 was evaluated at the protein level by western blotting with an antibody to the pATM/ATR substrate. The data are summarized in FIGS. 5A-5B.

Conclusion. The data show that inhibition of SMG1 by Compound 1 causes up-regulation of mutant p53. These data suggest that tumors with mutations in p53, especially those which lead to NMD transcripts, may be more responsive to Compound 1 treatment.

### 6.1.6 Xenograft Studies

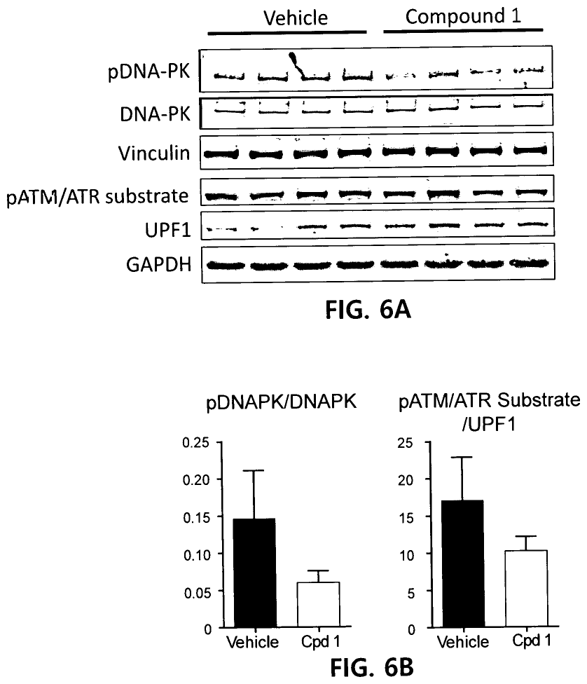

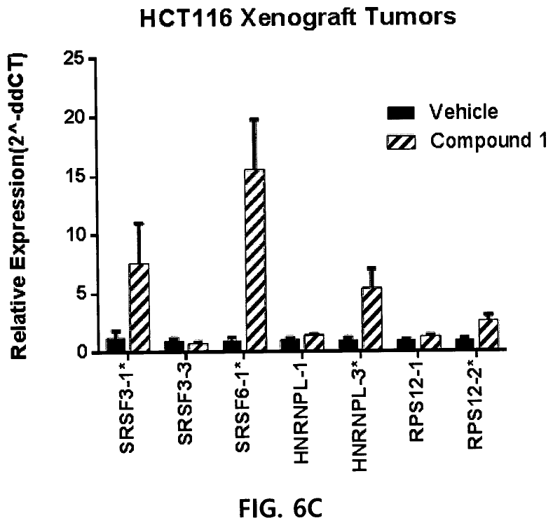

In vivo studies: All animal studies were performed under protocols approved by Institutional Animal Care and Use Committees. Animals were housed and xenograft studies were performed as previously published (See Mortensen DS F. K.-N., 2015). Inhibition of xenograft tumors was analyzed using Western blot and qPCR analysis as provided in FIGS. 6A-6C. Compound 1 (1-ethyl-7-(2-methyl-6-(1H-1,2,4-triazol-3-yl)pyridin-3-yl)-3,4-dihydropyrazino[2,3-b]pyrazin-2(1H)-one) was shown to inhibit SMG1 in vivo.

Conclusion. The data shows that Compound 1 inhibits SMG1, as evidenced by decreased pUPF1, and upregulation of NMD mRNAs and DNAPK, in xenograft tumors in vivo.

A number of references have been cited, the disclosures of which are incorporated herein by reference in their entirety. The embodiments disclosed herein are not to be limited in scope by the specific embodiments disclosed in the examples which are intended as illustrations of a few aspects of the disclosed embodiments and any embodiments that are functionally equivalent are encompassed by the present disclosure. Indeed, various modifications of the embodiments disclosed herein are in addition to those shown and described herein will become apparent to those skilled in the art and are intended to fall within the scope of the appended claims.

1. A method for treating or preventing an NMD-dependent tumor, the method comprising administering an effective amount of an NMD-inhibitor to a patient having an NMD-dependent tumor.

2. The method of claim 1, wherein the NMD-dependent tumor is an SMG1-dependent tumor.

3. The method of claim 1, wherein the NMD-inhibitor is an SMG1-inhibitor.

4. The method of claim 3, wherein the SMG1-inhibitor is 1-ethyl-7-(2-methyl-6-(1H-1,2,4-triazol-3-yl)pyridin-3-yl)-3,4-dihydropyrazino[2,3-b]pyrazin-2(1H)-one or a tautomer, solvate, hydrate, co-crystal, clathrate, polymorph, or pharmaceutically acceptable salt thereof.

5. (canceled)

6. The method of claim 1, wherein the NMD-dependent tumor is characterized by modulation of activity or expression of an NMD component.

7. The method of claim 4, wherein the NMD component is one or more of SMG1, UPF1, UPF2, UPF3b, eRF1, eRF3, SMG5, SMG6, SMG7, SMG8, or SMG9.

8. The method of claim 4, wherein the NMD component comprises a mutation.

9. The method of claim 6, wherein the modulation of an NMD component is an amplification, overexpression, or modulated activity of the NMD component.

10. The method of claim 6, wherein the NMD component is SMG1.

11. The method of claim 10, wherein the NMD-dependent tumor is characterized by modulation of activity of SMG1.

12. The method of claim 10, wherein the NMD-dependent tumor is characterized by modulation of expression of an SMG1-marker.

13. The method of claim 12, wherein the SMG1-marker comprises one or more of phospho-Upf1, Luc71, Neu1, Srsf3, Srsf6, Hnrnp1, Hnrnpk, Rps12, or Sf1, or a combination thereof.

14. The method of claim 12, wherein the SMG1-marker comprises one or more of phospho-Upf1, Neu1, Srsf3, Srsf6, Hnrnp1, Hnrnpk, Rps12, or Sf1, or a combination thereof.

15. The method of claim 1, further comprising administering an immuno-oncology treatment, a proteasome inhibitor, a premature termination codon (PTC) read-through compound, an NMD-inhibitor, or a splice factor inhibitor.

16. The method of claim 1, wherein the NMD-dependent tumor is characterized by dysregulated splicing of one or more cancer-associated genes.

17. The method of claim 16, wherein the cancer-associated gene is one or more of AIMP2, AR, BCL-X, BCRL11, BIRC5, BRAF, BRD4, CASP3, CD22, CD44, CDH17, CEACAM1, CLDN18, CPE, CXCL12, EGFR, ENHA, FAS, FGFR2, FN1, FPGS, HIF1A, IKZF1, KLF6, KLK8, MCL1, MDM2, MET, MKNK2, REST4, S6K1, SYK, TERT, TP53, VEGFA, or WFDC2, or a combination thereof.

18. (canceled)

19. The method of claim 1, wherein the NMD-dependent tumor is characterized by modulation of activity or expression of one or more splicing factors.

20. The method of claim 19, wherein the splicing factor comprises a mutation.

21. (canceled)

22. (canceled)

23. The method of claim 19, wherein the splicing factor is one or more of DDX5, DDX23, DDX46, DHX15, DHX16, DHX38, DHX8, DIMT1, EFTUD2, HNRNPF, HNRNPH1, HNRNPL, LUC7L2, LUC7L3, PRPF19, PRPF3, PRPF31, PRPF39, PRPF4, PRPF38A, PRPF40A, PRPF40B, PRPF6, PRPF8, SF1, SF3A1, SF3A2, SF3A3, SF3B1, SF3B14, SF3B2, SF3B3, SF3B4, SF3B5, SNRNP200, SNRNP27, SNRNP35, SNRNP40, SNRNP70, SNRPB, SNRPC, SNRPD1, SNRPD2, SNRPD3, SNRPE, SNRPF, SNRPG, SRSF1, SRSF10, SRSF2, SRSF3, SRSF5, SRSF6, SRSF7, U2AF1, U2AF2, ZRSR2, or a combination thereof.

24. The method of any claim 19, wherein the splicing factor is one or more of SF1, SF3A1, SF3A2, SF3A3, SF3B1, SF3B14, SF3B2, SF3B3, SF3B5, SRSF1, SRSF10, SRSF2, SRSF3, SRSF5, SRSF6, SRSF7, U2AF1, U2AF2, ZRSR2, or a combination thereof.

25. (canceled)

26. The method of claim 1, wherein the NMD-dependent tumor is characterized by dysregulated splicing due to modulation of activity or expression of one or more splicing regulators.

27. The method of claim 26, wherein the one or more splicing regulators are selected from the group consisting of SSFPQ, AFF2, CASC3, CCAR1, CDK12, CELF2, CHERP, DDX5, DDX39A, DHX9, DHX15, DHX35, GEMIN6, HNRNPC, HNRNPH, HSPA8, LSM6, MBNL1, NONO, PABPC1, PCBP1, PPIH, PRMT7, PPP2R1A, PRPF28B, PUF60, RBFOX2, RHEB, PSIP1, PRPF4, RBM10, RBM17, RBM25, RBMX, RNU6-1, RP9, SART1, SNU13, SMC1A, SMNDC1, SNRPA, SNRPA1, SNRPB2, SNRPB, SNRPD1, SNRPD2, SNRPF, SNRPN, SON, SRRM2, SCNM1, SRRM2, SYNCRIP, TCERG1, TXNL4A, TXNL4B, U2AF35, U2SURP, USP39, WBP11, and WT1.

28. The method of claim 26, wherein the one or more splicing regulators comprises a mutation.

29. (canceled)

30. (canceled)

31. The method of claim 1, wherein the NMD-dependent tumor is a breast tumor, ovarian tumor, papillary thyroid tumor, perivascular epithelioid cell tumor (PEComa), renal tumor, adenocystic carcinoma, chronic lymphocytic leukemia (CLL), myelodysplastic syndrome (MDS), skin melanoma, uveal melanoma, T-ALL, acute myelogenous leukemia (AML), chronic myelomonocytic leukemia (CMML), diffuse large B cell lymphoma (DLBCL), myelofibrosis, essential thrombocythemia, multiple myeloma, lung tumor, pancreatic tumor, prostate tumor, Wilms tumor, or glioblastoma.

32. (canceled)

33. (canceled)

34. (canceled)

35. A method for testing for responsiveness to an SMG1-inhibitor administered to a patient for treatment or prevention of an NMD-dependent tumor, the method comprising:
(a) measuring a level of expression or activity of an SMG1-marker in a biological sample obtained from the patient;
(b) administering a dosage amount of the SMG1-inhibitor to the patient;
(c) measuring a level of expression or activity of the SMG1-marker in a second biological sample obtained from the patient after the administration of the dosage amount; and
(d) comparing the levels of expression or activity of the SMG1-marker from the first and second biological samples;
wherein a modulation of the level of expression or activity of the SMG1-marker indicates an increased likelihood that the patient will be responsive to treatment with the SMG1-inhibitor.

36. The method of claim 35, wherein the dosage amount comprises an effective amount of the SMG1-inhibitor.

37. The method of claim 35, wherein the SMG1-marker comprises one or more of phospho-Upf1, Luc71, Neu1, Srsf3, Srsf6, Hnrnp1, Hnrnpk, Rps12, or Sf1, or a combination thereof.

38. The method of claim 35, wherein the SMG1-marker comprises one or more of phospho-Upf1, Neu1, Srsf3, Srsf6, Hnrnp1, Hnrnpk, Rps12, or Sf1, or a combination thereof.

39. (canceled)

40. A method for achieving one or more clinical endpoints associated with treating or preventing an NMD-dependent or SMG1-dependent tumor comprising administering an NMD-inhibitor or SMG1-inhibitor to a patient having an NMD-dependent or SMG1-dependent tumor.

41. The method of claim 40, wherein the clinical endpoint is selected from positive tumor response, complete response, partial response or stable disease, increased survival without tumor progression, inhibition of disease progression, inhibition of tumor growth, reduction of primary tumor, relief of tumor-related symptoms, inhibition of tumor secreted factors, delayed appearance of primary or secondary tumors, slowed development of primary or secondary tumors, decreased occurrence of primary or secondary tumors, slowed or decreased severity of secondary effects of disease, arrested tumor growth and regression of tumors, increased Time To Progression (TTP), increased Progression Free Survival (PFS), and increased Overall Survival (OS).

42. The method of claim 41, wherein the positive tumor response is inhibition of tumor growth or a reduction in tumor size.

43. (canceled)

44. (canceled)

45. (canceled)

46. (canceled)

47. (canceled)

48. (canceled)

49. A method for decreasing mortality of a patient having an NMD-dependent or SMG1-dependent tumor, the method comprising administering an effective amount of an NMD-inhibitor or SMG1-inhibitor to a patient having an NMD-dependent or SMG1-dependent tumor.

50. (canceled)

51. (canceled)

52. A method for treating or preventing an NMD-dependent tumor, comprising administering to a patient having an NMD-dependent tumor a SMG1-inhibitor in combination with one or more of an immuno-oncology treatment, a proteasome inhibitor, a premature termination codon (PTC) read-through compound, an NMD-inhibitor, or a splice factor inhibitor.

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. (canceled)

94. (canceled)

95. (canceled)

96. (canceled)

97. A method of testing for responsiveness to an NMD-inhibitor in a patient, wherein the method comprises screening a biological sample obtained from the patient for the presence of an NMD-marker, wherein the presence of the NMD-marker indicates an increased likelihood that the patient will be responsive to treatment with the NMD-inhibitor.

98. (canceled)

99. A method for testing for responsiveness of a patient to an SMG1-inhibitor administered to said patient for treatment or prevention of an NMD-dependent tumor, wherein the method comprises:
(a) measuring a level of expression or activity of an SMG1-marker in a biological sample obtained from said patient;
(b) measuring a level of expression or activity of said SMG1-marker in a second biological sample obtained from said patient, wherein said patient has been administered a dosage amount of said SMG1-inhibitor, optionally wherein said dosage amount comprises an effective amount of said SMG1-inhibitor; and
(c) comparing the levels of expression or activity of said SMG1-marker from said first and second biological samples;
wherein a modulation of the level of expression or activity of said SMG1-marker indicates an increased likelihood that said patient will be responsive to treatment with said SMG1-inhibitor.

100. The method of claim 97, wherein said NMD-inhibitor or SMG1-inhibitor is 1-ethyl-7-(2-methyl-6-(1H-1,2,4-triazol-3-yl)pyridin-3-yl)-3,4-dihydropyrazino[2,3-b]pyrazin-2(1H)-one or a tautomer, solvate, hydrate, co-crystal, clathrate, polymorph, or pharmaceutically acceptable salt thereof; and/or
wherein said NMD-marker or SMG1-marker comprises one or more of phospho-Upf1, Luc71, Neu1, Srsf3, Srsf6, Hnrnp1, Hnrnpk, Rps12, Sf1, or a combination thereof; and/or
wherein said patient has further been administered an immuno-oncology treatment, a proteasome inhibitor, a premature termination codon (PTC) read-through compound, an NMD-inhibitor, or a splice factor inhibitor.

101. (canceled)

102. (canceled)
